(19) United States
(12) Patent Application Publication
(10) Pub. No.: US 2022/0072095 A1
(43) Pub. Date: Mar. 10, 2022

(54) METHODS AND COMPOSITIONS FOR TREATING SKIN DISEASES

(71) Applicant: VANDERBILT UNIVERSITY, Nashville, TN (US)

(72) Inventors: Jack Jacek HAWIGER, Nashville, TN (US); Yan LIU, Nashville, TN (US); Ruth Ann VEACH, Brentwood, TN (US); Jozef ZIENKIEWICZ, Nashville, TN (US)

(21) Appl. No.: 17/275,764

(22) PCT Filed: Sep. 13, 2019
(86) PCT No.: PCT/US2019/051005

§ 371 Date: Mar. 12, 2021

Related U.S. Application Data
(60) Provisional application No. 62/731,394, filed on Sep. 14, 2018.
(60) Provisional application No. 62/733,997, filed on Sep. 20, 2018.

(51) Int. Cl. A61K 38/17; A61K 45/06

(52) U.S. Cl. CPC A61K 38/1709; A61K 45/06

(57) ABSTRACT

Disclosed are compositions and methods for treating skin diseases mediated by inflammation that is caused by microbial, autoimmune, allergic, metabolic, neoplastic, and physical factors and insults (wounds, burns, UV light and radiation). In one aspect, the compositions and methods disclosed herein can also be used to enhance clearance of microbes from infected skin and subcutaneous tissue, in a subject. Also disclosed herein are compositions and methods for reducing levels of stress-responsive transcription factors and metabolic transcription factors in a cell in a subject with inflammation-mediated skin diseases.

This application claims the benefit of U.S. Provisional Application No. 62/733,997, filed on Sep. 20, 2018 and U.S. Provisional Application No. 62/731,394, filed on Sep. 14, 2018, applications which are incorporated herein by reference in their entirety.

### I. BACKGROUND

Millions of people in the United States and around the world suffer from skin diseases. In the United States, almost 85 million Americans were seen by a physician for at least one skin disease in 2013. Half of 24 skin categories had a fatal outcome and the estimated direct health care cost of skin diseases was $46 billion. Inflammation is a fundamental mechanism of skin diseases caused by microbial, autoimmune, allergic, constitutive, metabolic, physical and neoplastic insults (see Table 1).

TABLE 1
Skin Diseases Mediated by Inflammation

| Type of | Cause of | Examples of Skin Diseases Mediated by a |
|---|---|---|
| Inflammation | Inflammation | Given Type of Inflammation |
| Microbial | Bacteria, Fungi, Mites, and | Abscess, Acne, Blepharitis, Candidiasis, |
| Inflammation | Viruses | Dermatophytic Infections of the Folds, Erysipelas, |
| | | Fistulas, Furunculosis, Genital Warts, Impetigo, |
| | | Seborrheic Dermatitis*, Moluscum Contagiosum, |
| | | Necrotizing Fasciitis, Paronychia, Purpura Fulminans, |
| | | Scabies, Shingles, Tinea Faciei |
| Autoimmune | Aberrant Autoimmune | Erythema Nodosum, Fistulas and Oral Ulcers in |
| Inflammation | Attack | Crohn Disease, Graft-Versus-Host Disease, |
| | | Pemphigus, Psoriasis, Scleroderma, Systemic Lupus |
| | | Erythematosus |
| Allergic | Allergens | Atopic Dermatitis/Eczema, Contact Dermatitis |
| Inflammation | | including Poison Ivy, Oak, and Sumac, Drug |
| | | Hypersensitivity Reactions, Insect Bites |
| Constitutive | Inborn Errors of Innate | Familial Mediterranean Fever, NEMO Mutation- |
| Inflammation | Immunity | Linked Intestinal and Skin Autinflammatory |
| | | Disease |
| Metabolic | Excessive Accumulation of | Seborrheic Dermatitis*, Gout (Tophi), Xanthelasma |
| Inflammation | Metabolites (e.g. sebum, | |
| | cholesteryl esters or uric acid | |
| | deposits) | |
| Physical | Trauma, Burns, or Radiation | Bedsores, Chemical, Electric and Thermal (scalding) |
| Inflammation | | Burns, Radiation Injury, Skin Aging |
| Neoplastic | Unknown | Mycosis Fungoides, Sezary Syndrome |
| Inflammation | Kaposi's Sarcoma- | Kaposi's Sarcoma |
| | Associated Herpesvirus | |
| | Human T-Cell Leukemia | Adult T cell Leukemia/Lymphoma with skin |
| | Virus-1 | erythematous and itchy plaques |

* Seborrheic Dermatitis is mediated by both Metabolic and Microbial Inflammation

Non-immune cells comprising skin keratinocytes, epithelial cells, and melanocytes form the outermost layer of the skin containing also hair follicles. These cells are subject to inflammatory insults. Another non-immune cell type, endothelial cells, comprise the innermost lining of small, medium and large blood vessels. These non-immune cells provide not only important “barrier” function, but also are the first line sentinels responsible for recognizing exogenous and endogenous causes of inflammation. Together with strategically located macrophages, dendritic cells, Natural Killer (NK) cells, and group 1, 2, and 3 innate lymphoid cells (ILC), non-immune cells alert immune cells to the presence of inflammation-causing irritants and modulate the inflammatory response. Immune cells comprise polymorphonuclear leukocytes, also known as granulocytes, divided into neutrophils, basophils, and eosinophils. Mononuclear phagocytes that also evolve from the myeloid progenitors encompass monocytes, macrophages, and dendritic cells. Lymphoid cells include B and T lymphocytes, natural killer T cells, and ILCss. Inflammatory insults such as microbial agents, allergens, autoantigens, excessive metabolites, and chemicals represented by phorbol-12-myristate-13-acetate (PMA also termed phorbol ester), evoke activation of signal transduction pathways that recruit stress-responsive transcription factors (SRTFs) and metabolic transcription factors (MTFs) such as Sterol regulatory Element Binding Proteins (SREBPs) 1 and 2 and Carbohydrate Regulatory Element Binding Proteins (CHREBPs). These proinflammatory and metabolic transactivators are ferried to the nucleus by transport shuttles termed importins alpha and beta (FIGS. 1 A and B). The proinflammatory signaling cascades culminate in the cell's nucleus and induce a wide-range reprogramming of the genome. MTFs are activated by overfeeding with dietary fats and sugars. As a result, multiple mediators of inflammation are produced. They attract immune cells that produce their own mediators of inflammation thereby reinforcing and perpetuating inflammatory response in the skin and surrounding tissue. Skin injury ensues. It is being sculpted by the type of inflammation (Table 1).

Among skin diseases mediated by microbial inflammation, abcess and furuncle (“a boil”) are most commonly caused by Staphylococcus aureus. These diseases can spread to other family members and become recurrent especially in patients with underlying conditions such as diabetes, obesity, and hematologic diseases. Seborrrrheic dermatitis that affects 1-3% of general population is associated with increased sebum production and chronic infection with Malassezia yeasts and Staphylococcus aureus. Other skin disease mediated by microbial inflammation such as impetigo and necrotizing fasciitis due to Streptococcus spp. are treated with pathogen-directed anti-microbial therapy. However, localized or systemic inflammation causes collateral damage to the skin structural integrity. In some cases, microbial inflammation impedes the action of anti-microbial therapy and perpetuates skin injury due to the action of the microbial virulence factors that are not inhibited by anti-microbials. These virulence factors, such as streptokinase, produced by Streptococci, relentlessly perpetuate damage to skin and subcutaneous tissues in genetically-prone individuals, as documented in necrotizing fasciitis. This rapidly progressing destruction of skin and underlying structures caused by the notorious “flesh-eating bacteria” is life-threathening and requires extensive skin grafting. Therefore, adjuvant anti-inflammatory therapy is urgently needed to counteract pathogen- and host-activated proteases responsible for the skin and subcutaneous tissue necrosis due to out-of-control microbial inflammation.

Autoimmune inflammation is caused by an aberrant autoimmune attack by the clones of autoreactive T lymphocytes that attack skin cells in psoriasis and the joint lining in psoriatic arthritis manifested by enthesitis and dactylitis. Autoreactive B lymphocytes that produce anti-DNA antibodies are associated with skin lesions and other organs dysfunction (e.g. cardiovascular system and kidneys) in lupus erythematosus. Autoreactive B and T cells usually persist due to their resistance to activation-induced cell death. Nuclear Transport Modifiers (NTMs), the class of anti-inflammatory peptides described in this Application, reversed the resistance of autoreactive T-cells to activation-induced cell death. The elimination of islet-infiltrating, autoreactive B and T lymphocytes is a prominent feature of the the NTM-modulated process of beta cell protection. In Crohn disease, especially in children, perioral and oral ulcers and perianal lesions that may include fissures, fistulae, or perinal abcesses may evolve into strictures and other complications.

Allergic contact dermatitis is a type IV hypersensitivity reaction to contact allergens. The induction of contact hypersensitivity (CHS) is biphasic: a sensitization phase and an elicitation phase. After the first allergen sensitization, dermal dendritic cells (DCs) that recognize and bind allergens migrate from the skin into the draining lymph nodes (LNs). Therein, they prime naive T cells. Allergen-specific T lymphocytes in the LNs are activated upon re-challenge with the same allergen, migrate, and infiltrate the site of skin challenge with allergen.

Skin diseases mediated by metabolic inflammation encompass acne and seborrheic dermatitis that are caused by accumulation of dying skin cells and the oily skin metabolite, sebum, around hair follicles. These sites become occluded causing redness, swelling, itching, the typical signs of inflammation. Metabolic skin inflammation is aggravated by secondary infections with the members of skin microbiome, such as Priopionibacterium acnes. Thus, increased sebum production is causing metabolic inflammation in the skin while some members of the skin microbiome, yeasts and bacteria, cause microbial inflammation that is frequently recurring. Therefore, combination treatment with topical antimicrobial agents and systemic therapy with drugs improving skin metabolism, e.g. isotretinoin, is used with limited success.

Skin diseases mediated by physical inflammation encompass accidental and surgical wounds, aging-related skin microvascular lesions (senile purpura), thermal, chemical, and electric burns, sunburns and radiation-induced skin burns. Critically injured patients who suffered trauma and burns displayed in their peripheral blood leukocytes a broad spectrum of activated genes that encode inflammatory cytokines and chemokines, signal transducers (cyclooxygenase and nitric oxide synthase) and cell adhesion molecules. Nuclear Transport Modifier (NTM) was effective in experimental model of traumatic brain injury. In addition to the transcriptional cascade mediated by NF-κB and linked to neuronal cell apoptosis that was prevented by NTM, other transcriptional cascades, in particular AP-1 and SREBP, are at play as well. It is plausible, that beneficial effect of NTM on traumatic brain injury can also be linked to its targeting of importin β1 thereby suppressing expression of SREBP1 as well as four other stress-responsive transcription factors (see FIGS. 1A and 1B). Photo-aging of the skin is caused by the cumulative effect of UV exposure and formation of reactive oxygen species and reactive nitrogen species. These insults induce chronic inflammation that contributes to the extracellular matrix degradation, especially breakdown of collagen and elastin.

Neoplastic inflammation of the skin is represented by cutaneous T cell lymphoma known as Mycosis Fungoides that also includes Cesary Syndrome. These two most common types of cutaneous T cell lymphoma are manifested by scaly, red rash in the body area not exposed to sun. It evolves into eczema-like rash followed by hardened lesions of the skin termed papuls. In Sezary Syndrome, generalized rash with plaques and small tumors is itchy, peeling, and painful. In contrast, another neoplastic skin disease mediated by microbial inflammation is Kaposi sarcoma. It is caused by Kaposi-associated herpes virus and by tumor cells-elicited inflammatory mediators. It afflicts not only patients with Acquired Immunodeficiency Syndrome but also in organ transplant recipients receiving immunosuppresants as well as in older men of Mediterranean descent and young men in Africa.

Many skin diseases mediated by inflammation are not adequately treated using conventional therapeutics. Steroidal anti-inflammatory drugs (e.g., hydrocortisone, prednisone, and methylprednisolone) have significant treatment-associated side effects such as skin thinning and delayed wound healing, muscle weakness, increased susceptibility to infections, cataracts, increased in intraocular pressure, stomach ulcers, and psychiatric disturbances. They also have significant metabolic side effects increasing blood glucose, blood lipids and body fat distribution. Some children are intolerant of steroidal anti-inflammatory drugs. Non-steroidal anti-inflammatory drugs (e.g., aspirin, ibuprofen, naproxen, CELEBREX®) may cause fluid retention leading to edema, kidney failure (primarily with chronic use), liver failure, ulcers and prolonged bleeding after an injury or surgery. Newer immunosuppressive drugs, tacrolimus and picrolimus (e.g. Elidel), that inhibit macrophilin-12 also known as FKBP-12, have a potential risk of lymphomas and skin cancer. Thus, there is a critical need for more effective therapeutics for preventing and treating inflammation-mediated diseases.

### II. SUMMARY

Disclosed are methods and compositions related to treating an inflammatory skin disorder.

In one aspect, disclosed herein are methods of treating/inhibiting/reducing an inflammatory skin disorder (such as, for example, a skin disorder caused by microbial agents, autoimmune process, allergic process, constitutive autoinflammatory process, metabolic process, neoplastic process, and/or physical factors and/or physical insults that include wounds, burns, UV radiation, gamma radiation) in a subject comprising administering to the subject a therapeutically effective amount of a composition comprising one or more Nuclear Transport Modifier (NTM) such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

Also disclosed herein are methods of any preceding aspect, further comprising administering to the subject an anti-microbial agent.

In one aspect, disclosed herein are methods of treating a wound comprising contacting the wound with a therapeutically effective amount of a composition comprising a Nuclear Transport Modifier (NTM) such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

Also disclosed herein are methods of reducing the healing time of a wound comprising contacting the wound with a therapeutically effective amount of a composition comprising a Nuclear Transport Modifier (NTM) such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

In one aspect, disclosed herein are medicated adhesive bandages, wound dressings, surgical drapes, sutures, salves, creams, or wound adhesives comprising a therapeutically effective amount of a composition comprising a Nuclear Transport Modifier (NTM) such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

Also disclosed herein are methods of treating/inhibiting/reducing an inflammatory skin disorder, treating a wound, and/or reducing the healing time of a wound of any preceding aspect, comprising administering to a subject with a skin disorder and/or wound the medicated adhesive bandages, wound dressings, surgical drapes, sutures, salves, creams, or wound adhesives of any preceding aspect.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIG. 1A presents a schematic showing that bacteria, fungi, viruses, allergens, and Phorbol Myristoyl Acetate (PMA), known inducer of proinflammatory signaling pathways to the cell's nucleus, evoke signal transduction and activation of proinflammatory stress responsive transcription factors (SRFTs). The nuclear transport of SRTFs is a pivotal checkpoint in genomic regulation of cell's response to inflammatory insults. The blockade with cell-penetrating NTM peptides reduces availability of SRTFs thereby interrupting the proinflammatory signaling cascades and calming activated genome. Thus, production of cytokines, chemokines, and adhesins as well as migration of immune cells and their adhesion are impeded thereby alleviating skin injury. Legend: NFAT (nuclear factor of activated T cells); AP-1 (Activator protein 1); NF-κB (Nuclear factor kappa B); NPC (nuclear pore complex); STAT1 (signal transducer and activator of transcription 1); Imp α5 (Importin alpha 5); Imp β1 (Importin beta 1); TNFα (tumor necrosis factor alpha); IL-1, IL-6, IL-10 and IL-17 (interleukin 1, 6, 10 and 17, respectively); MCP-1 (Monocyte Chemoattractant Protein-1).

FIG. 1B presents a schematic drawing in which Metabolic Transcription Factors are activated by overfeeding with dietary fats (SREBPs) and/or sugars-induced hyperglycemia (CHREBPs).

FIGS. 2A, 2B, 2C, and 2D show Hematoxylin and eosin (H & E) staining of paraffin-embedded skin biopsies demonstrating that increased inflammatory cell infiltration in response to Phorbol Myristoyl Acetate (PMA), known inducer of proinflammatory signaling pathways to the cell's nucleus, is reduced by dose-dependent NTM treatment. FIG. 2A shows vehicle only; FIG. 2B shows PMA+ vehicle; FIG. 2C shows PMA+ low dose NTM; and FIG. 2D shows PMA+ high dose NTM. Pictures from PMA-challenged mice are representative of 3 mice/group)

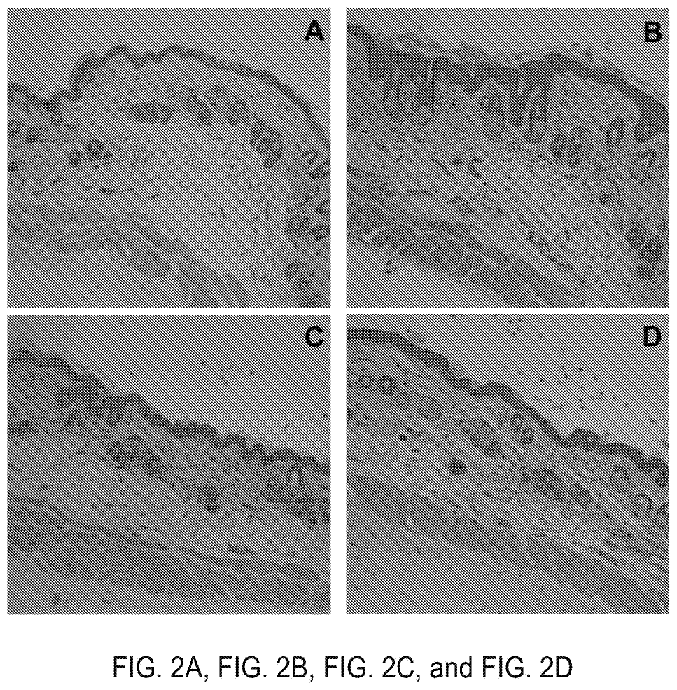

FIG. 3 shows ear thickness measurements of intact ears show that ear swelling due to increased microvascular permeability induced by PMA is attenuated by topical NTM treatment. Shown are mean±SEM from right ear of 3 mice/group (p value determined by repeated measures two way ANOVA comparing right ear measurements from mice treated with PMA+ vehicle to those treated with PMA+NTM).

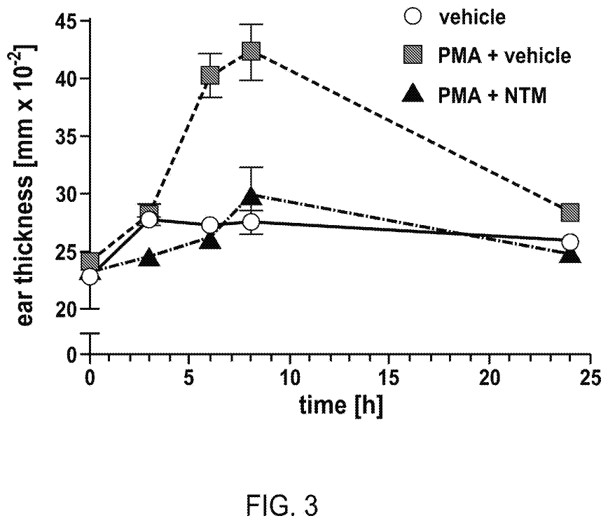

FIGS. 4A, 4B, and 4C show H & E staining of paraffin-embedded ear punch biopsies shows that increased swelling and cellular infiltration induced by PMA are reduced by NTM treatment 8 h post-challenge. FIG. 4A shows vehicle only; FIG. 4B shows PMA+ vehicle; and FIG. 4C shows PMA+NTM. Inset pictures of ears show redness and swelling induced by PMA after 6 h is absent in NTM-treated mice. Pictures are representative of 3 mice/group.

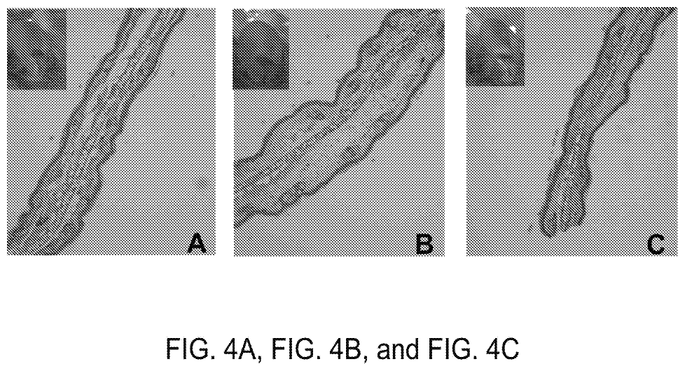

FIG. 5 shows that continuous NTM treatment does not impede skin wound healing and hair regrowth after repeated surgical trauma. ALZET Osmotic pumps loaded with NTM (cSN50.1 peptide, 10 mg in 100 ul of H₂O) were implanted under the shaved skin and replaced at weakly intervals. Healed skin wounds that were produced at the beginning of a 3-week experiment and reopened twice during weakly implantation of osmotic micropumps containing NTM or diluent show no apparent signs of infection or impeded healing. Thus, skin wound healing was not adversely affected by NTM treatment as compared to diluent. Significantly, no excessive scaring, infection or hair loss is apparent in NTM-treated animals.

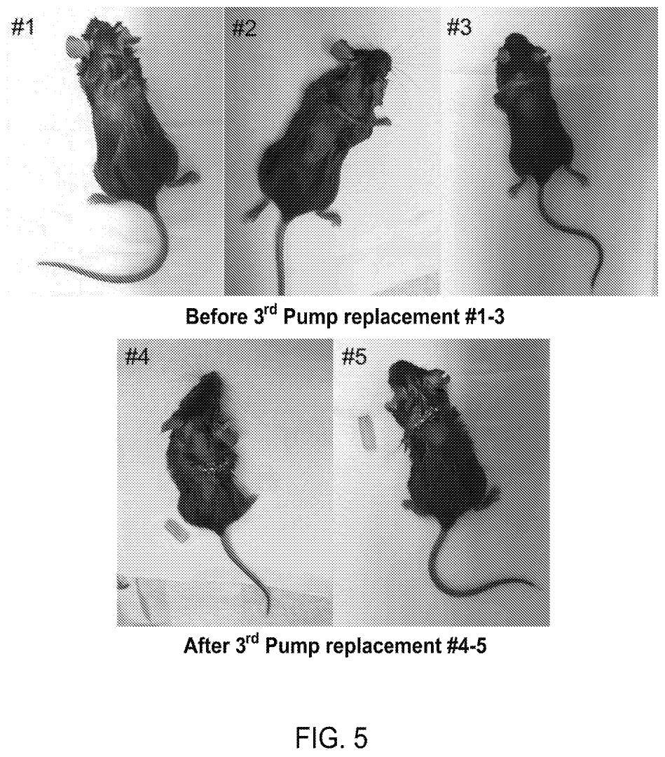

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. DEFINITIONS

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims, which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

The terms “patient,” “subject” and “individual” are used interchangeably herein, and mean an animal (e.g., mammalian (such as human, equine, bovine, ovine, porcine, canine, etc.), reptilian, piscine, etc.) to be treated, diagnosed and/or to obtain a biological sample from.

As used herein, “bind,” “binds,” or “interacts with” means that one molecule recognizes and adheres to a particular second molecule in a sample or organism, but does not substantially recognize or adhere to other structurally unrelated molecules in the sample. Generally, a first molecule that “specifically binds” a second molecule has a binding affinity greater than about 10⁸ to 10¹² moles/liter for that second molecule and involves precise “hand-in-a-glove” docking interactions that can be covalent and noncovalent (hydrogen bonding, hydrophobic, ionic, and van der Waals).

By the phrase “nuclear transport modifier” and “NTM” is meant a peptide that is capable of modulating entry of transcription factors into the nucleus. An example of a nuclear transport modifier is a 26-29 amino acid peptide derived from human nuclear factor kappa B1 nuclear localization sequence and from human Fibroblast Growth Factor 4 signal sequence hydrophobic region. This phrase is used interchangeably with the phrase “nuclear import inhibitor.”

In an NTM as described herein, any of the amino acid residues in the NTM sequence can be mutated and/or modified (i.e., to form mimetics) so long as the modifications do not affect the translocation-mediating function of the peptide. Thus, the word “peptide” includes mimetics and the word “amino acid” includes modified amino acids, unusual amino acids, D-form amino acids, etc.

By the phrases “importin beta-selective Nuclear Transport Modifier (NTM)” and “importin beta-selective NTM” is meant any NTM that binds to importin beta 1 and modifies its nuclear transport function while sparing a similar function of importins alpha and that modulates nuclear transport of at least one intracellular protein, e.g., an intracellular protein that regulates cell responses to metabolic and proinflammatory stimuli. Typically, the importin beta-selective NTM includes a peptide sequence that includes an SSHR domain derived from Signal Sequence Hydrophobic Region of Fibroblast Growth Factor 4 and a hydrophilic cargo to counterbalance hydrophobic properties of SSHR.

By the phrases “importin alpha-selective Nuclear Transport Modifier (NTM)” and “importin alpha-selective NTM” is meant any NTM that binds to major and/or minor binding pockets of one or more of importins alpha that recognize their own autoinhibitory regions or other proteins that bear a nuclear localization sequence (NLS) and are larger than approximately 45 kD (e.g., proinflammatory stress-responsive transcription factors) and that modulate nuclear transport of at least one intracellular protein, e.g., an intracellular protein that regulates cell responses to proinflammatory and metabolic stimuli. Typically, the importin alpha-selective NTM is the sequence of or a sequence derived from AAVALLPAVXLAXXAPCVQRKRQKLMPC (SEQ ID NO: 17), where X represents any amino acid from the group of hydrophobic or special amino acids (e.g., cysteine, glycine, and proline, non-natural amino acids) (e.g., cSN50.1 peptide).

As used herein, the phrases “nuclear import adaptor” and “nuclear transport adaptor” mean a cell component capable of mediating transport of a protein usually larger than 45 kD (e.g., a transcription factor) into the nucleus. An example of a nuclear transport adaptor is an importin also known as karyopherin.

As used herein, “protein” and “polypeptide” are used synonymously to mean any peptide-linked chain of amino acids, regardless of length or post-translational modification, e.g., glycosylation or phosphorylation.

By the term “gene” is meant a nucleic acid molecule that codes for a particular protein, or in certain cases, a functional or structural RNA molecule.

As used herein, a “nucleic acid” or a “nucleic acid molecule” means a chain of two or more nucleotides such as RNA (ribonucleic acid) and DNA (deoxyribonucleic acid).

The term “labeled,” with regard to a nucleic acid, protein, probe or antibody, is intended to encompass direct labeling of the nucleic acid, protein, probe or antibody by coupling (i.e., physically or chemically linking) a detectable substance (detectable agent) to the nucleic acid, protein, probe or antibody.

As used herein, the terms “therapeutic,” and “therapeutic agent” are used interchangeably, and are meant to encompass any molecule, chemical entity, composition, drug, cell(s), therapeutic agent, chemotherapeutic agent, or biological agent capable of preventing, ameliorating, or treating a disease or other medical condition. The term includes small molecule compounds, antisense reagents, siRNA reagents, antibodies, enzymes, peptides organic or inorganic molecules, cells, natural or synthetic compounds and the like.

As used herein, the term “treatment” is defined as the application or administration of a therapeutic agent to a patient or subject, or application or administration of the therapeutic agent to an isolated tissue or a cell from a patient or subject, who has a disease, a symptom of disease or a predisposition toward a disease, with the purpose to cure, heal, alleviate, relieve, alter, remedy, ameliorate, improve or affect the disease, the symptoms of disease, or the predisposition toward disease.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also, for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular NTM is disclosed and discussed and a number of modifications that can be made to a number of molecules including the NTM are discussed, specifically contemplated is each and every combination and permutation of NTM and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

### B. METHODS OF USE

Although compositions, kits, cells, and methods similar or equivalent to those described herein can be used in the practice or testing of the present invention, suitable compositions, kits, cells, and methods are described below. All publications, patent applications, and patents mentioned herein are incorporated by reference in their entirety. U.S. patent application Ser. No. 14/349,918, and U.S. Pat. No. 7,553,929, for example, are incorporated by reference in their entireties. In the case of conflict, the present specification, including definitions, will control. The particular embodiments discussed below are illustrative only and not intended to be limiting.

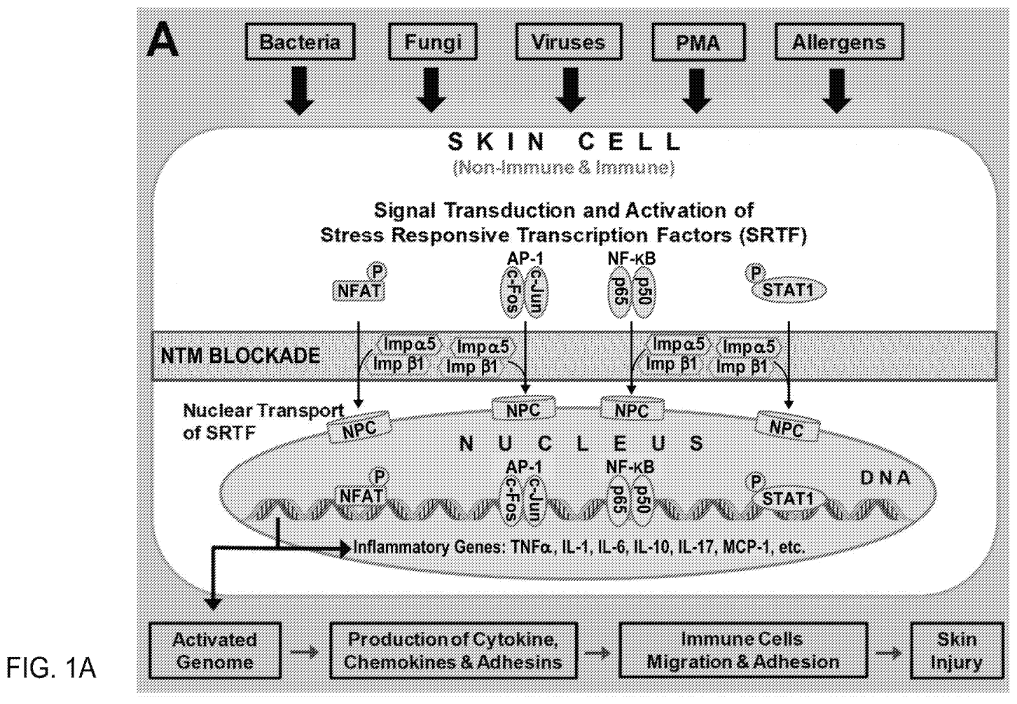

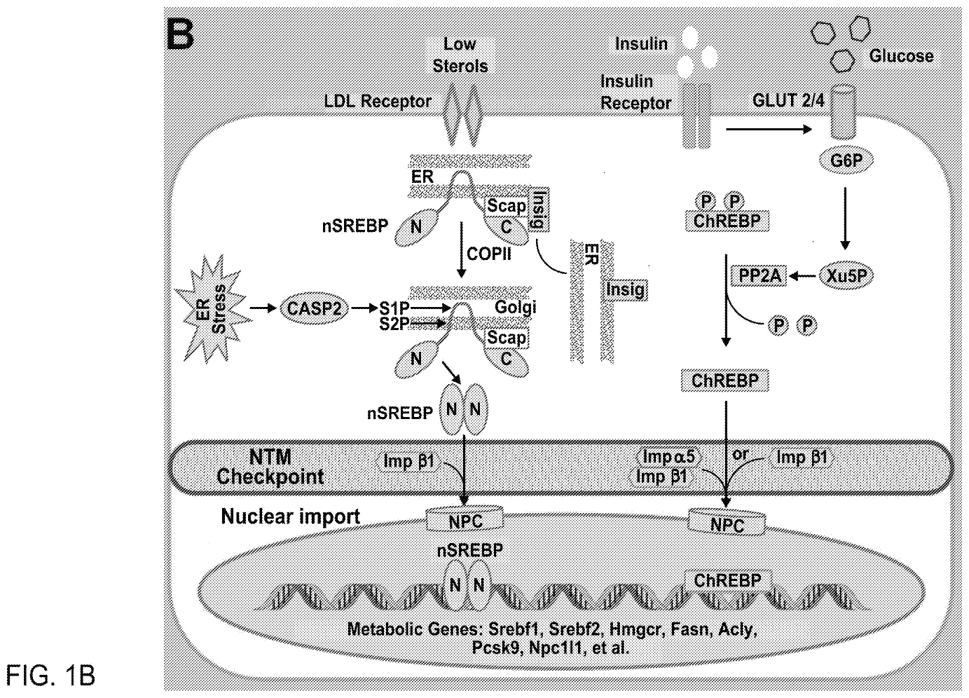

Small transcription factors (<45 kD), usually those regulating the housekeeping genes that encode cell survival factors, have free passage from the cytoplasm to the nucleus. In contrast, nuclear transport of transcription factors larger than 45 kD, such as SRTFs, is guided by one or more nuclear localization sequences (NLSs). These intracellular “zip codes” are displayed on SRTFs upon stimulation of immune and non-immune cells by microbial insults. NLSs are then recognized by nuclear transport adaptor proteins, importins/karyopherins alpha (Imp α) (see FIG. 1A). The stimulus-induced formation of SRTF and importins α complexes also encompasses importin beta 1 (Imp β1), which is recognized by nuclear pore proteins to allow translocation of the cargo to the nucleus. Until recently, nuclear transport has been targeted through the forced expression of genes that encode inhibitors of proinflammatory SRTFs, such as the degradation-resistant inhibitor of NF-κB termed IκBα. However, NF-κB is only one of multiple SRTFs that mediate signaling to the nucleus in response to infection. Other SRTFs, such as AP-1, STAT1 and NFAT, are also transported to the nucleus during the inflammatory response yet their nuclear transport is not impeded by IκBα; contrarily, the AP-1 pathway is activated. Targeting nuclear transport, a pivotal checkpoint integrating translocation of multiple transcription factors to the nucleus, can be a more efficient strategy than targeting signaling pathways of individual transcription factors. This concept was proven by design and development of NTMs.

NTMs target the nuclear transport shuttles, Imp α5 and Imp β1, that translocate SRTFs to the nucleus and control signal transduction pathways, which culminate in genomic reprogramming NTMs modulate signaling to the nucleus mediated by transcription factors that include but are not limited to NFκB, AP-1, NFAT, STAT1 that utilize importins alpha and beta heterodimer, or SREBP1a, SREBP1c, and SREBP2, that utilize solely importin beta for nuclear transport whereas ChREBP can utilize primarily importins alpha/beta heterodimer for nuclear translocation. SRTFs such as NFκB, AP-1, NFAT, STAT1 are transported to the nucleus in response to proinflammatory stimuli. In the nucleus, SRTFs activate genes that encode mediators of inflammation. Examples of NTMs include SN50, cSN50 and cSN50.1 described in more detail in the following paragraphs, as well as the sequences set forth in Table 2.

In recent preclinical studies, a highly soluble cell-penetrating NTM (cSN50.1), with dual specificity was used. This NTM has segments that bind both Imp α5, which recognizes NLS derived from NFκB1, and Imp β1, which recognizes the signal-sequence hydrophobic region (SSHR) derived from Fibroblast Growth Factor 4. SSHR also serves as a membrane translocating motif (MTM) to enable intracellular delivery of peptides and proteins through an ATP- and endocytosis-independent mechanism. This and other NTMs have been shown to inhibit nuclear translocation of SRTFs and metabolic transcription factors, Sterol Regulatory Element Binding Proteins (SREBPs) thereby reducing inflammatory responses, microvascular injury, apoptosis and hemorrhagic necrosis as well as correcting metabolic derangements (eg. hyperlipidemia, with a concomitant gain in survival, in models of lethal shock induced by bacterial toxins.

A novel form of immunotherapy that targets nuclear import as described herein can arrest inflammation-driven destruction of microbe-infected tissue and surrounding area of a given organ. With respect to microbial inflammation (such as, for example, acute inflammation, subacute inflammation, chronic inflammation, skin-specific inflammation, systemic inflammation), pro-inflammatory signaling initiated through stimulation of the principal receptors of innate immunity, Toll-like receptors (TLRs), is one mechanism that activates antigen-presenting cells (APCs). Reprograming of gene regulatory networks in response to a multitude of microbial insults is dependent on signaling to the host cell's nucleus comprising a fundamental process of microbial inflammation (see FIG. 1A for a depiction). Inhibiting nuclear transport at a common “checkpoint” located downstream of TLRs and cytokine receptors globally suppresses expression of inflammatory genes thereby calming the genomic storm and averting multiple organ injury.

Accordingly, in one aspect, disclosed herein are methods of reducing levels of a SRTF and metabolic transcription factors, Carbohydrate Regulatory Element Binding Proteins (ChREBPs), and Sterol Regulatory Element Binding Proteins (SREBPs) in a cell's nucleus at a site of inflammation in a subject with a skin disease, comprising administering to the subject a therapeutically effective amount of a composition comprising one or more NTMs.

It is understood and herein contemplated that by reducing the levels of SRTF and metabolic transcription factors, ChREBPS, and SREBPs, in a cell's nucleus, the disclosed NTM can reduce, inhibit, and/or prevent skin inflammation (such as, for example, a skin disorder caused by microbial agents that induce microbial inflammation, autoimmune process, autoinflammatory process, metabolic disorder, neoplastic disorder, and/or physical factors and/or physical insults that are mediated by inflammation, as displayed in Table 1, and including, but not limited to contact dermatitis, psoriasis, systemic lupus erythematosus, bullous dermatitis, “flesh-eating disease”, seborrheic dermatitis, atopic dermatitis, and graft-versus-host disease). Accordingly, described herein is a method of treating, inhibiting, reducing, and/or preventing skin diseases (such as, for example, mediated by microbial inflammation, autoimmune inflammation, allergic inflammation, metabolic inflammation, neoplastic inflammation, and physical inflammation as exemplified in Table 1 comprising administering to the subject with the skin disease mediated by microbial inflammation a composition comprising NTM in combination with one or more anti-microbial agents.

In one aspect, the method for reducing levels of SRTF, ChREBPs and SREBPs (such as, for example, ChREBPα, ChREBPβ, SREBP1a, SREBP1c, SREBP2) in a cell, methods treating, inhibiting, reducing, and/or preventing skin inflammation includes administering a therapeutically effective amount of a composition comprising one or more NTM to the mammalian subject. Administration of the composition decreases inflammation by attenuating expression of at least one stress-responsive transcription factor-regulated gene, or at least one ChREBPs and/or one SREBPs-regulated gene. Thus, the effective dose is an amount effective for reducing importin alpha-mediated nuclear translocation of at least one stress response SRTF or one ChREBPs and reducing skin inflammation (such as, for example, a skin disorder caused by microbial agents that induce microbial inflammation, autoimmune process, autoinflammatory process, metabolic disorder, neoplastic disorder, and/or physical factors and/or insults that are mediated by inflammation, including, but not limited to contact dermatitis, psoriasis, systemic lupus erythematosus, bullous dermatitis, “flesh-eating disease”, seborrheic dermatitis, atopic dermatitis, and graft-versus-host disease) in the mammalian subject. Similarly, the effective dose is an amount effective for reducing importin beta-mediated nuclear translocation of at least one metabolic transcription factors, SREBP and reducing a skin inflammation in the mammalian subject. The NTM may bind to importin alpha, to importin beta, or to both importin alpha and importin beta.

An important aspect of the NTM exemplified by cSN50.1 peptide and its congeners is their ability to reach the site of infection and the infected host cell in the skin, as well as other myeloid, lymphoid, and non-lymphoid organs. The mechanism of intracellular delivery of this class of cell-penetrating peptides has been elucidated and an endocytosis-independent process of crossing the plasma membrane mediated by the membrane-translocating motif (MTM), which is based on the SSHR derived from Kaposi FGF, has been documented (Veach et al. (2004) J Biol Chem 279: 11425-11431). The amphipathic helix-based structure of SSHR facilitates its insertion directly into the plasma membrane and the tilted transmembrane orientation permits the translocation of the NTM through the phospholipid bilayer of the plasma membrane directly to the interior of the cell without perturbing membrane integrity. This mechanism explains the efficient delivery of SSHR-guided cargo across the plasma membrane of multiple cell types involved in microbial inflammation, autoimmune inflammation, allergic inflammation, metabolic inflammation, neoplastic inflammation, and physical inflammation that mediate skin diseases.

The NTMs disclosed herein are derived from N50-containing NTMs (SN50, cSN50, and cSN50.1) that are comprised of a hydrophilic N50 motif patterned on the nuclear localization sequence (NLS) region of the NFκB1/p50 subunit (see Table 2) fused to a motif from the signal SSHR of human fibroblast growth factor 4. The SSHR allows peptides to cross the plasma membrane by an ATP- and endosome-independent mechanism, and the N50 motif was designed to bind to importins α during stimulus-initiated signaling and thereby limit docking of NLS-bearing SRTFs to their adaptor proteins and reduce nuclear import of activated STRFs. Any mimetics, derivatives, or homologs of SN50, cSN50, and cSN50.1 may be used in the compositions, methods, and kits disclosed herein.

TABLE 2
Amino Acid Sequences of Peptides Used

| NTM | SSHR | NLS | SEQ ID NO: |
|---|---|---|---|
| N50 | | VQRKRQKLMP | 10 |
| N50M | | VQRDEQKLMP | 11 |
| cN50.1 | | CVQRKRQKLMPC | 12 |
| SN50 | AAVALLPAVLLALLAP | VQRKRQKLMP | 13 |
| SSHR-1 | AAVALLP | | 14 |
| SSHR-2 | AVLLALLAP | | 15 |

N50-sequence derived from the NLS region of NFκB1/p50;
N50M-sequence of control peptide with KR to DE mutation
(bolded); cN50.1-sequence of cyclized version of
N50 just as cSN50.1 is a cyclized version of SN50.
Hydrophobic regions of the SSHR domain are distinguished
from the cluster of basic amino acids (NLS). NTM
indicates nuclear transport modifier; SSHR, signal
sequence hydrophobic region; NLS, nuclear localization
sequence.

SN50 is a fragment linked peptide combining the SSHR of the Kaposi fibroblast growth factor (K-FGF) and the NLS of the p50 subunit of NFκB1. Any mimetics, derivatives, or homologs of SN50 may be used in the compositions, methods, and kits disclosed herein. The sequence of SN50 is AAVALLPAVLLALLAPVQRKRQKLMP (SEQ ID NO: 13). Generation and use of SN50 is described in U.S. Pat. No. 7,553,929.

cSN50 is a fragment-designed cyclic peptide combining the hydrophobic region of the Kaposi fibroblast growth factor signal sequence with the nuclear localization signal (NLS) of the p50-NFKB1 and inserting a cysteine on each side of the NLS to form an intrachain disulfide bond. The amino acid sequence of cSN50 is AAVALLPAVLLALLAPCYVQRKRQKLMPC (SEQ ID NO: 1). Any mimetics, derivatives, or homologs of cSN50 may be used in the compositions, methods, and kits disclosed herein. Methods of making and using cSN50 are described, for example, in U.S. Pat. Nos. 7,553,929 and 6,495,518. These patents are incorporated herein by reference in their entireties.

cSN50.1 is a cyclized peptide having the sequence of cSN50 with the exception that the tyrosine at position 18 of cSN50, adjacent to the first cysteine, has been removed. Methods of making and using cSN50 are described, for example, in U.S. Pat. Nos. 7,553,929 and 6,495,518. The amino acid sequence of cSN50.1 is AAVALLPAVLLALLAPCVQRKRQKLMPC (SEQ ID NO: 2). The tyrosine at position 18 was removed from the sequence of cSN50 to increase solubility. cSN50 is soluble at levels of ranging from 2.0 mg/mL to 40 mg/mL depending on the method of synthesis and purification whereas cSN50.1 is soluble at levels of at least 100 mg/ml. Any mimetics, derivatives, or homologs of cSN50.1 may be used in the compositions, methods, and kits disclosed herein. cSN50.1 is also encompassed by SEQ ID NO: 3, SEQ ID NO: 4 and SEQ ID NO: 5. Additional examples of NTMs include fragment-designed and synthesized peptides in which cargo is incorporated as two, rather than one, modules or cargos derived from intracellular proteins other than NFκB 1. Such additional examples include the sequences of SEQ ID NO: 6, SEQ ID NO: 7, SEQ ID NO: 8, and SEQ ID NO: 9.

Accordingly, the NTM for use in the disclosed methods of treating, inhibiting, reducing, and/or preventing inflammatory skin diseases including, but not limited to microbial disease, autoimmune disease, autoinflammatory disease, metabolic disorder, neoplastic disorder, or physical injuries that are mediated by inflammation may be, for example, an NTM having the sequence Xaa Xaa Xaa Xaa Leu Leu Pro Xaa Xaa Leu Leu Ala Leu Leu Ala Pro Xaa Xaa Xaa Gln Arg Lys Arg Gln Lys Xaa Xaa Xaa Xaa (SEQ ID NO: 3), wherein Xaa is any amino acid or is absent. For example, the Nuclear Transport Modifier can have the sequence Xaa Xaa Xaa Xaa Leu Leu Pro Xaa Xaa Leu Leu Ala Leu Leu Ala Pro Cys Xaa Xaa Gln Arg Lys Arg Gln Lys Xaa Xaa Xaa Cys, where Xaa is any amino acid or is absent (SEQ ID NO: 4). As another example, the Nuclear Transport Modifier can have the sequence Xaa Xaa Xaa Xaa Leu Leu Pro Xaa Xaa Leu Leu Ala Leu Leu Ala Pro Cys Xaa Gln Arg Lys Arg Gln Lys Xaa Xaa Xaa Cys, where Xaa is any amino acid or is absent (SEQ ID NO: 5). In one embodiment, the Nuclear Transport Modifier is cSN50.1 having the sequence set forth in SEQ ID NO: 2. In another example of an NTM, the NTM has the sequence Xaa Xaa Xaa Xaa Leu Leu Pro Xaa Xaa Leu Leu Ala Val Leu Ala Pro Xaa Xaa Xaa Gln Arg Lys Arg Gln Lys Xaa Xaa Xaa Xaa, where Xaa is any amino acid or is absent (SEQ ID NO: 6). In yet another example, the NTM has the sequence Ala Ala Val Ala Leu Leu Pro Ala Val Leu Leu Ala Val Leu Ala Pro Cys Val Gln Arg Lys Arg Gln Lys Leu Met Pro Cys (SEQ ID NO: 7). In a further example, the NTM has the sequence Xaa Xaa Xaa Xaa Leu Leu Pro Xaa Xaa Leu Leu Ala Val Leu Ala Pro Xaa Xaa Xaa Gln Arg Asp Glu Gln Lys Xaa Xaa Xaa Xaa, where Xaa is any amino acid or is absent (SEQ ID NO: 8). In another example, the NTM has the sequence Ala Ala Val Ala Leu Leu Pro Ala Val Leu Leu Ala Val Leu Ala Pro Cys Val Gln Arg Asp Glu Gln Lys Leu Met Pro Cys (SEQ ID NO: 9).

1. Compositions for Treating Skin Diseases and Disorders Associated with Inflammation in a Subject

Compositions (e.g., pharmaceutical compositions) described herein for treating diseases associated with inflammation include a pharmaceutically acceptable carrier and at least one importin beta-selective and/or at least one importin alpha-selective NTM in an amount effective for modifying (e.g., decreasing) entry into the nucleus of at least one transcription factor that includes but is not limited to NFκB, AP-1, NFAT, STAT1, ChREBPα, ChREBPβ, SREBP1a, SREBP1c, and SREBP2, that utilize importins alpha and/or beta for nuclear transport, and treating or preventing the disease. For example, entry of at least one SREBP into the nucleus is reduced. As mentioned above, NTMs modulate signaling to the nucleus mediated by transcription factors that include but are not limited to NFκB, AP-1, NFAT, STAT1 that utilize importins alpha and beta heterodimer, SREBP1a, SREBP1c, and SREBP2, that utilize solely importin beta for nuclear transport whereas ChREBP can utilize primarily importins alpha for nuclear translocation. In this example, the importin beta-selective NTM reduces nuclear translocation of the nuclear forms of SREBP1a, SREBP1c, and SREBP2. Any suitable importin beta-selective NTM may be used. Examples of importin beta-selective NTMs include but are not limited to peptide sequences that include an SSHR domain listed in Table 3 below and a cargo listed in Table 2 above. One example of such an importin beta-selective NTM is AAVALLPAVLLALLAPVQRDEQKLMP (SEQ ID NO: 40) (i.e., a peptide sequence having the SSHR domain of AAVALLPAVLLALLAP (SEQ ID NO: 17) and the cargo of VQRDEQKLMP (SEQ ID NO: 11) as listed in Table 3 below). Additional examples of peptides designed to inhibit interaction of importin alpha with importin beta necessary for the formation of their heterodimer include AAVALLPAVLLALLAPRRRRIEVNVELRKAKK (SEQ ID NO: 18) (referred to as SIBB in Table 3), AAVALLPAVLLALLAPRRRRIEVNVELRKAKKDD (SEQ ID NO: 19) (referred to as SI-1 in Table 3). AAVALLPAVLLALLAPRRQRNEVVVELRKNKRDE (SEQ ID NO: 20) (referred to as SI-3 in Table 3), AAVALLPAVLLALLAPRRHRNEVTVELRKNKRDE (SEQ ID NO: 21) (referred to as SI-4 in Table 3), AAVALLPAVLLALLAPRRRREEEGLQLRKQKREE (SEQ ID NO: 22) (referred to as SI-5 in Table 3), AAVALLPAVLLALLAPRRRREEEGIQLRKQKREQ (SEQ ID NO: 23) (referred to as SI-7 in Table 3) and AAVALLPAVLLALLAPCTEMRRRRIEVC (SEQ ID NO: 24) (referred to as cSIB in Table 3). The examples of peptides designed to be specific inhibitors of importins alpha include AAVALLPAVLLALLAPVELRKAKKDDQMLKRRNVSSF (SEQ ID NO: 25) (referred to as SARI in Table 3), AAVALLPAVLLALLAPVELRKNKRDEHLLKRRNVPHE (SEQ ID NO: 26) (referred to as SAR3 in Table 3), AAVALLPAVLLALLAPVELRKNKRDEHLLKKRNVPQE (SEQ ID NO: 27) (referred to as SAR4 in Table 3), AAVALLPAVLLALLAPLQLRKQKREEQLFKRRNVATA (SEQ ID NO: 28) (referred to as SAR5 in Table 3), AAVALLPAVLLALLAPIQLRKQKREQQLFKRRNVELI (SEQ ID NO: 29) (referred to as SAR7 in Table 3), AAVALLPAVLLALLAPCVELRKAKKDDQC (SEQ ID NO: 30) (referred to as cSAR1-C in Table 3), AAVALLPAVLLALLAPCVELRKNKRDEHC (SEQ ID NO: 31) (referred to as cSAR3-C in Table 3), AAVALLPAVLLALLAPCLQLRKQKREEQC (SEQ ID NO: 32) (referred to as cSAR5-C in Table 3), AAVALLPAVLLALLAPCIQLRKQKREQQC (SEQ ID NO: 33) (referred to as cSAR7-C in Table 3), AAVALLPAVLLALLAPCQMLKRRNVSSFC (SEQ ID NO: 34) (referred to as cSAR1-N in Table 3), AAVALLPAVLLALLAPCHLLKRRNVPHEC (SEQ ID NO: 35) (referred to as cSAR3-N in Table 3), AAVALLPAVLLALLAPCHLLKKRNVPQEC (SEQ ID NO: 36) (referred to as cSAR4-N in Table 3), AAVALLPAVLLALLAPCQLFKRRNVATAC (SEQ ID NO: 37) (referred to as cSAR5-N in Table 3), and AAVALLPAVLLALLAPCQLFKRRNVELIC (SEQ ID NO: 38) (referred to as cSAR7-N in Table 3). It is to be understood that any derivatives and/or analogues of these sequences are encompassed by the invention.

In one embodiment, an NTM as described herein has the sequence AAVALLPAVXLAXXAPVELRKNKRDEHLLKRRNVPHE (SEQ ID NO: 39). Additional NTMs include SEQ ID NOs: 1-9, 13, and 16-41. It is to be understood that any derivatives and/or analogues of these sequences are encompassed by the invention.

An NTM as described herein may be an inhibitor of an importin alpha 3 interaction with importin beta.

The SI-3 sequence (see Table 3) is designed to block an interaction between importin alpha and importin beta. Hence, this peptide is a cell-penetrating inhibitor of an importin alpha and importin beta interaction. It is to be understood that any derivatives and/or analogues of this sequence is encompassed by the invention.

TABLE 3
Peptide sequences

| | SSHR^{§} | Cargo | SEQ ID NO: | Comments |
|---|---|---|---|---|
| SM12 | AAVALLPAVLLALLAP | VQRDEQKLMP | 40 | Importin beta- |
| | | | | selective inhibitor |
| | | | | (binding studies) |
| SIBB | AAVALLPAVLLALLAP | RRRRIEVNVELRKAKK | 18 | Inhibitor of Imp |
| | | | | alpha 1-importin |
| | | | | beta interaction |
| SI-1 | AAVALLPAVLLALLAP | RRRRIEVNVELRKAKKDD | 19 | Inhibitor of Imp |
| | | | | alpha 1-importin |
| | | | | beta interaction |
| SI-3 | AAVALLPAVLLALLAP | RRQRNEVVVELRKNKRDE | 20 | Inhibitor of Imp |
| | | | | alpha 3-importin |
| | | | | beta interaction |
| SI-4 | AAVALLPAVLLALLAP | RRHRVENTVELRKNKRDE | 21 | Inhibitor of Imp |
| | | | | alpha 4-importin |
| | | | | beta interaction |
| SI-5 | AAVALLPAVLLALLAP | RRRREEEGLQLRKQKREE | 22 | Inhibitor of Imp |
| | | | | alpha 5-importin |
| | | | | beta interaction |
| SI-7 | AAVALLPAVLLALLAP | RRRREEEGIQLRKQKREQ | 23 | Inhibitor of Imp |
| | | | | alpha 7-importin |
| | | | | beta interaction |
| SAR1 | AAVALLPAVLLALLAP | VELRKAKKDDQMLKRRNVSSF | 25 | Imp alpha 1-specific |
| SAR3 | AAVALLPAVLLALLAP | VELRKNKRDEHLLKRRNVPHE | 26 | Imp alpha 3-specific |
| SAR4 | AAVALLPAVLLALLAP | VELRKNKRDEHLLKKRNVPQE | 27 | Imp alpha 4-specific |
| SAR5 | AAVALLPAVLLALLAP | LQLRKQRKEEQLFKRRNVATA | 28 | Imp alpha 5-specific |
| SAR7 | AAVALLPAVLLALLAP | IQLRKQKREQQLFKRRNVELI | 29 | Imp alpha 7-specific |
| cMN50.1 | AAVALLPAVXLAXXAP | CVQRKRQKLMPC | 17 | Imp alpha 5-selective |
| cSN50.1β | AAVALLAPVLLALLAP | CVQRDEQKLMPC | 16 | Imp beta-selective |
| | | | | (cell culture and |
| | | | | preclinical studies) |
| cSIB | AAVALLPAVLLALLAP | CTEMRRRRIEVC | 24 | Inhibitor of Imp |
| | | | | alpha 1-inportin |
| | | | | beta interaction |
| cSAR1-C | AAVALLPAVLLALLAP | CVELRKAKKDDQC | 30 | Imp alpha 1-specific |
| | | | | Proximal to C-terminal |
| cSAR3-C | AAVALLPAVLLALLAP | CVELRKNKRDEHC | 31 | Imp alpha 3-specific |
| | | | | Proximal to C-terminal |
| cSAR5-C | AAVALLAPVLLALLAP | CLQLRKQKREEQC | 32 | Imp alpha 5-specific |
| | | | | Proximal to C-terminal |
| cSAR7-C | AAVALLPAVLLALLAP | CIQLRKQKREQQE | 33 | Imp alpha 7-specific |
| | | | | Proximal to C-terminal |
| cSAR1-N | AAVALLPAVLLALLAP | CQMLKRRNVSSFC | 34 | Imp alpha 1-specific |
| | | | | Proximal to N-terminal |
| cSAR3-N | AAVALLPAVLLALLAP | CHLLKRRNVPHEC | 35 | Imp alpha 3-specific |
| | | | | Proximal to N-terminal |
| cSAR4-N | AAVALLPAVLLALLAP | CHLLKKRNVPQEC | 36 | Imp alpha 4-specific |
| | | | | Proximal to N-terminal |
| cSAR5-N | AAVALLPAVLLALLAP | CQLFKRRNVATAC | 37 | Imp alpha 5-specific |
| | | | | Proximal to N-terminal |
| cSAR7-N | AAVALLPAVLLALLAP | CQLFKRRNVELIC | 38 | Imp alpha 7-specific |
| | | | | Proximal to N-terminal |

^{§}Signal Sequence Hyrdophobic Region (SSHR)
“Cargo” comprises sequences of functionally active hydrophilic motifs (fragments) listed as
linear or cyclized peptides through addition of cysteine at the amino- and carboxy-termini
of respective linear peptides. Both linear and cyclized sequences are fused to hydrophobic
membrane translocation motif denoted SSHR.

In one aspect disclosed herein are methods of treating, inhibiting, reducing, and/or preventing inflammatory skin disorder (such as, for example, a skin disorder caused by microbial agents that induce microbial inflammation, autoimmune process, autoinflammatory process, metabolic disorder, neoplastic disorder, and/or physical factors and/or insults that are mediated by inflammation, including, but not limited to contact dermatitis, psoriasis, systemic lupus erythematosus, bullous dermatitis, “flesh-eating disease”, seborrheic dermatitis, atopic dermatitis, and graft-versus-host disease) in a subject comprising administering to the subject an anti-microbial agent and a composition comprising one or more NTMs including, but not limited to SN50 having the sequence set forth in SEQ ID NO: 1 or cSN50.1 having the sequence set forth in SEQ ID NO: 2, cSN50.1 beta having the sequence set forth in SEQ ID NO: 16, or any of the NTMs disclosed herein having the amino acid sequence set forth in SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. In one aspect the NTM can be cSN50.1 beta comprising the amino acid sequence AAVALLPAVLLALLAPCVQRDEQKLMPC (SEQ ID NO: 16). cSN50.1 beta is a cyclized peptide having the sequence of cSN50.1 with the exception that the lysine at the position 21 has been replaced by aspartic acid and the arginine residue at the position of 22 has been replaced by glutamic acid.

Accordingly, described herein is a composition for treating an inflammatory skin disease or disorder (e.g., autoimmune, autoinflammatory, microbial, metabolic, neoplastic, and posttraumatic skin disease) in a subject. The composition includes a pharmaceutically acceptable carrier and at least one (e.g., one, two, three, etc.) importin beta-selective NTM including an SSHR domain and a cargo that does not bind to any importin alpha, or at least one (e.g., one, two, three, etc.) importin alpha-selective NTM, in an amount effective for modifying entry of at least one (e.g., one, two, three, etc.) transcription factor (e.g., NFκB, AP-1, NFAT, STAT1, SREBP1a, SREBP1c, and SREBP2, and ChREBPα and ChREBń) into a cell's (e.g., a mammalian cell's) nucleus and for treating the inflammatory disease or disorder. The at least one importin alpha-selective NTM is a peptide or compound that binds to one or more binding pockets of an importin alpha and that modulates nuclear transport of at least one intracellular protein. Modifying entry of at least one transcription factor into a cell's nucleus includes inhibiting entry of the at least one transcription factor into the cell's nucleus. The at least one importin beta-selective NTM can have an amino acid sequence from the group of: SEQ ID NOs: 2 and 6 (e.g., AAVALLPAVLLALLAPVQRDEQKLMP (SEQ ID NO: 40) (referred to as SM12 in Table 3). The at least one importin alpha-selective NTM can have, for example, the amino acid sequence AAVALLPAVXLAXXAPCVQRKRQKLMPC (SEQ ID NO: 41). The composition can be administered with a corticosteroid or a non-steroidal anti-inflammatory agent. In another embodiment, the composition can further include a corticosteroid or a non-steroidal anti-inflammatory agent. The non-steroidal anti-inflammatory agent can be, for example, acetaminophen or ibuprofen or calcineurin inhibitor.

Also described herein is a method of treating or preventing inflammation in a mammalian subject (e.g., a human subject having a skin disease mediated by allergic, autoimmune, metabolic, microbial, posttraumatic or neoplastic inflammation). The method includes administering a composition including a pharmaceutically acceptable carrier and at least one importin beta-selective NTM including an SSHR domain and a cargo to the mammalian subject in an amount effective for modifying entry of at least one transcription factor (e.g., NFκB, AP-1, NFAT, STAT1, SREBP1a, SREBP1c, and SREBP2, and ChREBPα and ChREBPβ) into a cell's nucleus and for treating or preventing inflammation in the mammalian subject. In the method, the at least one importin beta-selective NTM binds to and inhibits the activity of at least one importin beta. Modifying entry of at least one transcription factor into a cell's nucleus includes inhibiting entry of the at least one transcription factor into the cell's nucleus. Administration of the composition generally results in inhibition of at least one signaling pathway associated with the inflammation. The at least one importin beta-selective NTM can have an amino acid sequence from the NTM sequences disclosed herein. The composition can be administered by any suitable route, e.g., topically, orally, intravenously, or subcutaneously.

Yet further described herein is a method of treating or preventing skin disease mediated by inflammation in a mammalian subject. The method includes administering a composition including a pharmaceutically acceptable carrier and at least one agent that inhibits an interaction between at least one importin alpha (e.g., importin alpha 1, importin alpha 3, importin alpha 4, importin alpha 5 and importin alpha 7), and at least one importin beta and that modulates nuclear transport of at least one intracellular protein, to the mammalian subject in an amount effective for modifying entry of at least one transcription factor into a cell's nucleus and for treating or preventing inflammation in the mammalian subject. Typically, the at least one agent binds specifically to the at least one importin alpha and is an importin alpha-selective inhibitor.

2. Methods of Treating Skin Disorders

In one aspect, disclosed herein are methods of treating/inhibiting/reducing an inflammatory skin disorder (such as, for example, a skin disorder caused by microbial agents that induce microbial inflammation, autoimmune process, autoinflammatory process, metabolic disorder, neoplastic disorder, and/or physical factors and/or insults that are mediated by inflammation, including, but not limited to contact dermatitis, psoriasis, systemic lupus erythematosus, bullous dermatitis, “flesh-eating disease”, seborrheic dermatitis, atopic dermatitis, and graft-versus-host disease) or inflammatory response caused by a skin insults in a subject comprising administering to the subject a therapeutically effective amount of a composition comprising one or more NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

As noted herein, inflammatory skin disorders can be caused by any number on insults including, but not limited to a skin disorder caused by a microbial infection (i.e., microbial disease). It is understood and herein contemplated that inflammation is a mechanism of disease caused by infection (“microbial insult”). An inflammatory skin disorder caused by a microbial insult evolves from innate immune response to an infection due to a microbe such as, for example, a virus, bacterium, fungus, or parasite. Thus, the microbial injury caused by microbial virulence factors is aggravated by the host-produced inflammatory mediators that impede the clearance of invading microbes and add insult to organ's injury. It is understood and herein contemplated that the inflammation and its end stage, necrosis of the skin and its underlying structures can result from any microbial insult elicited by known (or unknown) virulence factors and microbial antigens. Accordingly, in one aspect, disclosed herein are methods of treating an inflammatory skin disorder; wherein the inflammatory skin disorder is caused by a microbial disease such as, for example, a virus, bacterium, fungus, and/or parasite. Adjuvant anti-inflammatory therapy is urgently needed to counteract pathogen- and host-activated proteases responsible for the skin and subcutaneous tissue necrosis due to out-of-control microbial inflammation. Such adjuvant therapy is based on anti-inflammatory and cytoprotective action of NTMs. These cell-penetrating peptides suppress host-produced mediators of inflammation responsible for massive apoptosis and hemorrhagic necrosis of the liver and dramatically improve the clearance of invading bacteria in the lungs and other organs. Accordingly, disclosed herein are methods of treating, inhibiting, reducing, and/or preventing skin disease mediated by microbial inflammation in a subject comprising administering to a subject a therapeutically effective amount of an anti-microbial agent and a composition comprising one or more NTM (such as, for example, SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

In one aspect, disclosed herein are methods of treating an inflammatory skin disorder; wherein the inflammatory skin disorder is caused by a viral infection, such as, for example, an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

Also disclosed herein are methods of treating an inflammatory skin disorder; wherein the inflammatory skin disorder is caused by a bacterial infection, wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Nocardia asteroides, other Nocardia species, Legionella pneumophila, other Legionella species, Bacillus anthracis, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Vibrio vulnificus, Capnocytophaga canimorsus, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species. In some instances, the bacteria causing the bacterial infection is not Bacillus anthracis.

In one aspect, disclosed herein are methods of treating an inflammatory skin disorder; wherein the inflammatory skin disorder is caused by a fungal infection, wherein the fungal infection is an infection with a fungi selected from the group consisting of Candida albicans, Malassezia yeasts, Cryptococcus neoformans, Histoplasma capsulatum, Aspergillus fumigatus, Coccidiodes immitis, Paracoccidioides brasiliensis, Blastomyces dermitidis, Pneumocystis carnii, Penicillium marneffi, and Alternaria alternata.

Also disclosed herein are methods of treating an inflammatory skin disorder; wherein the inflammatory skin disorder is caused by a parasitic infection, wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Strongyloides stercoralis, Trichobilharzia regenti, Trichinella spiralis, Trichinella britovi, Trichinella nelsoni, Trichinella nativa, and Entamoeba histolytica.

It is understood and herein contemplated that while addressing the inflammatory skin disorder may alleviate symptoms of inflammatory disorder or alleviate the skin disorder caused by the microbial infection, the methods and NTMs disclosed herein will not remove the causative microbe (although such clearance could be driven by a properly regulated host immune response). It is understood and herein contemplated that any method of treating an inflammatory skin disorder comprising administering a composition comprising any of the NTM disclosed herein can further comprise the administration of an anti-microbial agent. Examples of anti-microbial agents include any antibiotics, antibodies, small molecules, and functional nucleic acids (siRNA, RNAi, anti-sense oligonucleotides), that directly attack the infecting microbe or alter host conditions rendering the host system inhospitable to the microbe. Such agents include, but are not limited to Abacavir, Acyclovir, Adefovir, Amantadine, Amprenavir, Ampligen, Arbidol, Atazanavir, Atripla, Balavir, Cidofovir, Combivir, Dolutegravir, Darunavir, Delavirdine, Didanosine, Docosanol, Edoxudine, Efavirenz, Emtricitabine, Enfuvirtide, Entecavir, Ecoliever, Famciclovir, Fomivirsen, Fosamprenavir, Foscarnet, Fosfonet, Ganciclovir, Ibacitabine, Imunovir, Idoxuridine, Imiquimod, Indinavir, Inosine, Lamivudine, Lopinavir, Loviride, Maraviroc, Moroxydine, Methisazone, Nelfinavir, Nevirapine, Nexavir, Nitazoxanide, Norvir, Oseltamivir, Peginterferon alfa-2a, Penciclovir, Peramivir, Pleconaril, Podophyllotoxin, Raltegravir, Ribavirin, Rimantadine, Ritonavir, Pyramidine, Saquinavir, Sofosbuvir, Stavudine, Telaprevir, Tenofovir, Tenofovir disoproxil, Tipranavir, Trifluridine, Trizivir, Tromantadine, Truvada, Valaciclovir, Valganciclovir, Vicriviroc, Vidarabine, Viramidine, Zalcitabine, Zanamivir, Zidovudine, Clofazimine; Dapsone; Capreomycin; Cycloserine; Ethambutol(Bs); Ethionamide; Isoniazid; Pyrazinamide; Rifampicin; Rifabutin; Rifapentine; Streptomycin; Arsphenamine; Chloramphenicol(Bs); Fosfomycin; Fusidic acid; Metronidazole; Mupirocin; Platensimycin; Quinupristin/Dalfopristin; Thiamphenicol; Tigecycline(Bs); Tinidazole; Trimethoprim(Bs); aminoglycosides such as, for example, Amikacin, Gentamicin, Kanamycin, Meropenem, Neomycin, Netilmicin, Tobramycin, Paromomycin, Streptomycin, Spectinomycin, Nitazoxanide, Melarsoprol Eflornithine, Metronidazole, Tinidazole, Miltefosine, Mebendazole, Pyrantel pamoate, Thiabendazole, Diethylcarbamazine, Ivermectin, Niclosamide, Praziquantel, Albendazole, Praziquantel, Rifampin, Amphotericin B, Fumagillin, Amphotericin B, Candicidin, Filipin, Hamycin, Natamycin, Nystatin, Rimocidin, Bifonazole, Butoconazole, Clotrimazole, Econazole, Fenticonazole, Isoconazole, Ketoconazole, Luliconazole, Miconazole, Omoconazole, Oxiconazole, Sertaconazole, Sulconazole, Tioconazole, Albaconazole, Efinaconazole, Epoxiconazole, Fluconazole, Isavuconazole, Itraconazole, Omadacycline, Posaconazole, Propiconazole, Ravuconazole, Terconazole, Voriconazole, Abafungin, Anidulafungin, Caspofungin, Micafungin, Aurones, Benzoic acid, Ciclopirox, Flucytosine, Griseofulvin, Haloprogin, Tolnaftate, Undecylenic acid, Crystal violet, Balsam of Peru, Orotomide, Miltefosine; ansamycins, such as, for example, geldanamycin, rifaximin, herbimycin; Carbapenems, such as, for example, Ertapenem, Doripenem, Imipenem/Cilastatin, and Meropenem; Cephalosporins, such as, for example, Cefadroxil, Cefazolin, Cephradine, Cephapirin, Cephalothin, Cefalexin, Cefaclor, Cefoxitin, Cefotetan, Cefamandole, Cefmetazole, Cefonicid, Loracarbef, Cefprozil, Cefuroxime, Cefixime, Cefdinir, Cefditoren, Cefoperazone, Cefotaxime, Cefpodoxime, Ceftazidime, Ceftibuten, Ceftizoxime, Moxalactam, Ceftriaxone, Cefepime, Ceftaroline fosamil, and Ceftobiprole; Glycopeptides, such as, for example Teicoplanin, Vancomycin, Telavancin, Dalbavancin, and Oritavancin; Lincosamides(Bs), such as, for example, Clindamycin and Lincomycin; Lipopeptides, such as, for example, Daptomycin; Macrolides(Bs), such as, for example, Azithromycin, Clarithromycin, Erythromycin, Roxithromycin, Telithromycin, and Spiramycin; Monobactams, such as, for example, Aztreonam; Nitrofurans, such as, for example, Furazolidone and Nitrofurantoin(Bs); Oxazolidinones(Bs), such as, for example, Linezolid, Posizolid, Radezolid, and Torezolid; Penicillins, such as, for example, Amoxicillin, Ampicillin, Azlocillin, Dicloxacillin, Flucloxacillin, Mezlocillin, Methicillin, Nafcillin, Oxacillin, Penicillin G, Penicillin V, Piperacillin, Penicillin G, Temocillin, and Ticarcillin; Polypeptides, such as, for example, Bacitracin, Colistin, and Polymyxin B; Quinolones/Fluoroquinolones, such as, for example, Ciprofloxacin, Enoxacin, Gatifloxacin, Gemifloxacin, Levofloxacin, Lomefloxacin, Moxifloxacin, Nadifloxacin, Nalidixic acid, Norfloxacin, Ofloxacin, Trovafloxacin, Grepafloxacin, Sparfloxacin, and Temafloxacin; Sulfonamides(Bs), such as, for example, Mafenide, Sulfacetamide, Sulfadiazine, Silver sulfadiazine, Sulfadimethoxine, Sulfamethizole, Sulfamethoxazole, Sulfanilimide (archaic), Sulfasalazine, Sulfisoxazole, Trimethoprim-Sulfamethoxazole (Co-trimoxazole) (TMP-SMX), and Sulfonamidochrysoidine (archaic); Tetracyclines(Bs), such as, for example, Demeclocycline, Doxycycline, Metacycline, Minocycline, Omadacycline, Oxytetracycline, and Tetracycline; monoclonal antibodies such as, for example, Actoxumab, Atidortoxumab, Berlimatoxumab, Bezlotoxumab, Cosfroviximab, Edobacomab, Felvizumab, Firivumab, Foravirumab, Larcaviximab, Motavizumab, Navivumab, Panobacumab, Palivizumab, Porgaviximab, CR6261, Rafivirumab, Pagibaximab, Obiltoxaximab, Ibalizumab, Regavirumab, Rmab, Sevirumab, Rivabazumab pegol, Tefibazumab, Suvratoxumab, and Tuvirumab; and checkpoint inhibitors; Pembrolizumab, Nivolumab, Atezolizumab, Avelumab, Durvalumab, pidilizumab, AMP-224, AMP-514, PDR001, cemiplimab, and Ipilimumab.

In one aspect, it is understood and herein contemplated that the inflammatory skin disorder treated using the methods and NTMs disclosed herein can be caused by an autoimmune disease. Autoimmune diseases are set of diseases, disorders, or conditions resulting from an adaptive immune response (autoreactive T cell and/or B cell response) against the host organism. In such conditions, either by way of mutation or other underlying cause, the host T cells and/or B cells and/or antibodies are no longer able to distinguish host cells, their constituents, and extracellular proteins from non-self-antigens and attack host cells (or their products) bearing an antigen for which they are specific. For example, autoreactive T lymphocytes that attack skin cells in psoriasis and the joint lining in psoriatic arthritis manifested by enthesitis and dactylitis. Autoreactive B lymphocytes that produce anti-DNA antibodies are associated with skin lesions and other organs dysfunction (eg cardiovascular system and kidneys) in lupus erythematosus. Autoreactive B and T cells usually persist due to their resistance to activation-induced cell death. Fortunately, they can be reduced or eliminated by treatment with NTM peptides in experimental model of autoimmune disease. Examples of autoimmune diseases that can cause an inflammatory skin disorder include, but are not limited to Contact Dermatitis, Graft-Versus-Host Disease, Pemphigus, Psoriasis, Rosacea, Scleroderma, Systemic Lupus Erythematosus, Achalasia, Acute disseminated encephalomyelitis, Acute motor axonal neuropathy, Addison's disease, Adiposis dolorosa, Adult Still's disease, Agammaglobulinemia, Alopecia areata, Amyloidosis, Ankylosing spondylitis, Antiphospholipid syndrome, Autoimmune angioedema, Autoimmune dysautonomia, Autoimmune urticaria, Behcet's disease, Bullous pemphigoid, Eosinophilic Granulomatosis (EGPA), Cicatricial pemphigoid, Cogan's syndrome, Cold agglutinin disease, Dermatitis herpetiformis, Dermatomyositis, Eosinophilic fasciitis, Erythema nodosum, Essential mixed cryoglobulinemia, Fibromyalgia, Granulomatosis with Polyangiitis, Graves' disease, Henoch-Schonlein purpura (HSP), Herpes gestationis or pemphigoid gestationis (PG), Hidradenitis Suppurativa (HS) (Acne Inversa), Lichen planus, Lichen sclerosus, Lupus nephritis, Lupus vasculitis, Lyme disease chronic, Microscopic polyangiitis (MPA), Mixed connective tissue disease (MCTD), Mooren's ulcer, Neonatal Lupus, Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

It is understood that not all inflammatory skin disorders resulting from attack by the host immune system involve the adaptive immune response. In some instances, the innate immune response (i.e., NK cells, macrophage, dendritic cells, and innate lymphoid cells) are constitutively activated and so produced inflammatory mediators attack the host cells. Diseases where the host innate immune response attacks host cells is referred to as an “autoinflammatory disease.” In one aspect, disclosed herein are methods of treating an inflammatory skin disorder in a subject comprising administering to the subject a therapeutically effective amount of a composition comprising a Nuclear Transport Modifier (NTM); wherein the inflammatory skin disorder is caused by an autoinflammatory disorder. Examples of autoinflammatory disorder that can cause the inflammatory skin disorders treated by the disclosed methods include, but are not limited to Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

In one aspect, it is understood and herein contemplated that metabolic disorders can underly the inflammation that results in an inflammatory skin disorder or inflammatory symptoms on the skin. As metabolic inflammation depends on nuclear transport of at least three classes of transcription factors SREBPs and ChREBPs, and proinflammatory SRTFs, NTM peptides that target signaling pathways mediated by these transcription factors (see FIGS. 1 A and B) are highly likely to be effective in these skin diseases. Accordingly, disclosed herein are methods of treating an inflammatory skin disorder, wherein the inflammatory skin disorder is caused by a metabolic syndrome or disease. In one aspect, the systemic or localized metabolic disorder can be selected from the group consisting of seborrheic acne, Gout, Skin Aging, Xanthelasma, metabolic syndrome, diabetes mellitus, obesity, Gaucher's disease, Phenylketonuria (PKU), Maple syrup urine disease (MSUD), fatty liver, hypercholesterolemia, hypertriglyceridemia, hyperthyroidism, hypothyroidism, dyslipidemia, hypolipidemia, and galactosemia.

It is understood and herein contemplated that inflammatory skin disorders can be caused by uncontrolled proliferation of certain types of skin cells or skin-infiltrating cells (i.e., neoplastic disorders and cancers). Thus, for example, disclosed herein are methods of treating inflammatory skin disorder comprising administering to a subject with an inflammatory skin disorder a therapeutically effective amount of a composition comprising a NTM, wherein the inflammatory skin disorder is caused by uncontrolled proliferation (such as, for example, a neoplastic disorder or cancer). In one aspect, disclosed herein are methods of treating an inflammatory skin disorder caused by a neoplastic disorder or a cancer, wherein the neoplastic disorder or cancer is selected from the group consisting of Mycosis Fungoides, Sezary Syndrome, Kaposi's Sarcoma, Adult T cell Leukemia/Lymphoma, PTEN hamartoma syndrome, Familial adenomatous polyposis, Tuberous sclerosis complex, Von Hippel-Lindau disease, ovarian teratomas, meningiomas, osteochondromas, B cell lymphoma, T cell lymphoma, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, and pancreatic cancer. In some instances, such as skin T-cell lymphoma, treatment involves extracorporeal exposure of blood to UV source with appropriate sensitizing agent. NTM peptides can be added to such a therapeutic system.

It is well established that physical injury through abrasion, puncture, laceration, contusion, blunt force trauma, ischemia, surgery, aging, aging caused by exposure to ultraviolet (UV) light, bedsores, transplant, sunburn, chemical burn, electrical burn, high temperature burn, low temperature burn can produce an inflammatory response. Some of these responses can either result in inflammation that manifests on the skin or an inflammatory skin disorder. Accordingly, disclosed herein are methods of treating an inflammatory skin disorder comprising administering to a subject with an inflammatory skin disorder a therapeutically effective amount of a composition comprising a NTM, wherein the inflammatory skin disorder is caused by physical injury. In one aspect, the physical injury can be selected from the group consisting of abrasion, puncture, laceration, contusion, blunt force trauma, ischemia, surgery, aging, aging caused by exposure to UV light, bedsores, transplant, sunburn, electrical burn, chemical burn, high temperature burn, low temperature burn.

The methods disclosed herein involve treating inflammatory skin disorders or symptoms from other inflammatory insults on the skin. It is understood and herein contemplated that many treatments of inflammatory conditions will involve the treatment of a wound. Thus, in one aspect, disclosed herein are methods of treating a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is further understood, that by treating a wound with a therapeutically effective amount of a composition comprising a NTM not only will the wound be treated, but the time needed for the healing process can be reduced compared to untreated wounds. Thus, disclosed herein are methods of reducing the healing time of a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

In one aspect, it is understood and herein contemplated that one way to treat a wound is through administration of the NTM subcutaneously, intramuscularly, intravenously, topically (such as, for example, through the use of salves, creams, and/or ointments), but also by impregnating bandages, dressing, sutures, drapes, surgical adhesive, and/or staples with the NTM. Thus, in one aspect, disclosed herein are medicated adhesive bandages, wound dressings, surgical drapes, sutures, salves, creams, or wound adhesives comprising a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is understood and herein contemplated that the medicated adhesive bandages, wound dressings, surgical drapes, staples, sutures, salves, creams, or wound adhesives disclosed herein can be used in conjunction with any of the disclosed methods of treatment. Thus, in one aspect, disclosed herein are methods of treating/inhibiting/reducing an inflammatory skin disorder (including, but not limited to inflammatory skin disorders caused by microbial disease, autoimmune disease, autoinflammatory disorder, metabolic disorder, neoplastic disorder, or physical insults that are mediated by inflammation), treating a wound, and/or reducing the healing time of a wound comprising administering to a subject with a skin disorder and/or wound the medicated adhesive bandages, wound dressings, surgical drapes, staples, sutures, salves, creams, or wound adhesives disclosed herein.

3. Methods of Treating Physical Factors and/or Physical Insults

Many inflammatory conditions result from physical injuries mediated by inflammation (such as, for example abrasion, puncture, laceration, contusion, blunt force trauma, ischemia, hemorrhagic stroke, surgery, transplant, bedsores, electric burn, sunburn, chemical burn, high temperature burn, low temperature burn, radiation injury, and skin aging). As noted above, the NTMs disclosed herein can target the nuclear transport shuttles, Imp α5 and Imp β1, that translocate SRTFs and SREBPs to the nucleus and control signal transduction pathways, which culminate in genomic reprogramming Thus, the novel forms of immunotherapy disclosed herein that targets nuclear import as described herein can arrest inflammation-driven destruction associated with these physical injuries. Accordingly, in one aspect, disclosed herein are methods of treating inflammation caused by physical injury (such as, for example, abrasion, puncture, laceration, contusion, blunt force trauma, ischemia, hemorrhagic stroke, surgery, transplant, sunburn, chemical burn, high temperature burn, low temperature burn) comprising administering to a subject with a physical injury a therapeutically effective amount of a composition comprising an NTM (such as, for example, a composition comprising an NTM an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41).

It is understood and herein contemplated that many inflammatory conditions resulting from inflammatory injury or physical injuries mediated by inflammation (such as, for example abrasion, puncture, laceration, contusion, blunt force trauma, ischemia, hemorrhagic stroke, surgery, transplant, bedsores, electric burn, sunburn, chemical burn, high temperature burn, low temperature burn, radiation injury, and skin aging), said treatments will involve the treatment of a wound. Thus, in one aspect, disclosed herein are methods of treating a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is further understood, that by treating a wound with a therapeutically effective amount of a composition comprising a NTM not only will the wound be treated, but the time needed for the healing process can be reduced compared to untreated wounds. Thus, disclosed herein are methods of reducing the healing time of a wound comprising contacting the wound with a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. In some aspect, the NTM can be administered orally, topically, intravenously, and/or a medicated adhesive bandage, wound dressing, surgical drape, suture, salve, cream, or wound adhesive comprising a therapeutically effective amount of a composition comprising a Nuclear Transport Modifier (NTM).

In one aspect, it is understood and herein contemplated that one way to treat a wound is through administration of the NTM subcutaneously, intramuscularly, intravenously, topically (such as, for example, through the use of salves, creams, and/or ointments), but also by impregnating bandages, dressing, sutures, drapes, surgical adhesive, and/or staples with the NTM. Thus, in one aspect, disclosed herein are medicated adhesive bandages, wound dressings, surgical drapes, sutures, salves, creams, lotions, or wound adhesives comprising a therapeutically effective amount of a composition comprising a NTM such as, for example, an NTM that comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41. It is understood and herein contemplated that the medicated adhesive bandages, wound dressings, surgical drapes, staples, sutures, salves, creams, or wound adhesives disclosed herein can be used in conjunction with any of the disclosed methods of treatment. Thus, in one aspect, disclosed herein are methods of treating/inhibiting/reducing a physical injury mediated by inflammation (including, but not limited to inflammatory skin disorders caused by physical insults that are mediated by inflammation), treating a wound, and/or reducing the healing time of a wound comprising administering to a subject with a skin disorder and/or wound the compositions comprising administering to the subject a therapeutically effective amount of a composition comprising a NTM and/or any medicated adhesive bandages, wound dressings, surgical drapes, staples, sutures, salves, lotions, creams, or wound adhesives disclosed herein.

4. Methods of Treating or Preventing Inflammatory Disorders in a Mammalian Subject

A typical method of treating or preventing an inflammatory disorder in a mammalian subject includes administering a composition including at least one importin alpha-selective NTM or at least one importin beta-selective NTM including an SSHR domain and a cargo, including peptides listed in Tables 2 and 3, to the mammalian subject in an amount effective for reducing importin alpha- and/or importin beta-mediated nuclear translocation of at least one transcription factor, and reducing inflammation in the mammalian subject. In the methods disclosed herein, the NTM reduces importin alpha-mediated nuclear translocation of SRTFs that respond to inflammatory stress and/or reduces importin alpha- or beta-mediated nuclear translocation of transcription factors that respond to metabolic stress, e.g., ChREBP and SREBP transcription factors by binding to importin alpha and to importin beta, respectively. Any suitable NTM can be used, e.g., one or more of the sequences disclosed herein, i.e., SEQ ID NOs: 1-9, 13, and 16-41 and/or derivatives and/or analogues thereof. The composition may be administered via any suitable route, e.g., orally, topically, intravenously, or subcutaneously. The therapeutic methods of the invention in general include administration of a therapeutically effective amount of a composition described herein to a subject (e.g., animal) in need thereof, including a mammal, particularly a human

5. Pharmaceutical Carriers/Delivery of Pharmaceutical Products

Compositions, e.g., pharmaceutical compositions, described herein for treating skin inflammation disorders (such as, for example, acute inflammation, subacute inflammation, chronic inflammation, organ-specific inflammation, systemic inflammation, and/or sepsis) including, but not limited to microbial disease, autoimmune disease, autoinflammatory disorder, metabolic disorder, neoplastic disorder, or physical factors and/or physical insults that are mediated by inflammation or an inflammatory skin disorder caused by microbial disease, autoimmune disease, autoinflammatory disorder, metabolic disorder, neoplastic disorder, or physical factors and/or physical insults that are mediated by inflammation) in a subject (e.g., a human subject) include a therapeutically effective amount of a NTM (such as cSN50, cSN50.1, cSN50.1 beta, or a NTM as set forth in SEQ ID NO: 3, SEQ ID NO: 4, SEQ ID NO: 5, SEQ ID NO: 6, SEQ ID NO: 7, SEQ ID NO: 8, and/or SEQ ID NO: 9) sufficient for treating inflammation disorders (such as, for example, acute inflammation, subacute inflammation, chronic inflammation, organ-specific inflammation, systemic inflammation, and/or purpura fulminans in sepsis) including, but not limited to skin disorder caused by microbial disease, autoimmune disease, autoinflammatory disorder, metabolic disorder, neoplastic disorder, or physical factors and/or physical insults that are mediated by inflammation. Similarly, compositions described herein for treating skin inflammation in a subject (e.g., a human subject) include a therapeutically effective amount of a NTM (such as cSN50, cSN50.1, cSN50.1 beta, or a NTM as set forth in SEQ ID NO: 3, SEQ ID NO: 4, SEQ ID NO: 5, SEQ ID NO: 6, SEQ ID NO: 7, SEQ ID NO: 8, and/or SEQ ID NO: 9) sufficient for reducing nuclear levels of a SRTF and SREBPs in a subject with an inflammation disorder (such as, for example, acute inflammation, subacute inflammation, chronic inflammation, organ-specific inflammation, systemic inflammation, and/or sepsis) including, but not limited to skin disorder caused by microbial disease, autoimmune disease, autoinflammatory disorder, metabolic disorder, neoplastic disorder, or physical factors and/or physical insults that are mediated by inflammation and a pharmaceutically acceptable carrier.

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, subcutaneous injection, by intraperitoneal injection, transdermally, extracorporeally, topically or the like, including topical intranasal administration or administration by inhalant. As used herein, “topical intranasal administration” means delivery of the compositions onto any dermal or exposed mucosal surface and can comprise delivery by creams, lotions, salves, wound adhesives, adhesive bandage, wound dressing, surgical drape, suture, spraying mechanism or droplet mechanism, or through aerosolization. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, an appropriate amount can be determined by one of ordinary skill in the art using only routine experimentation given the teachings herein.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that treats skin inflammatory disorders including, but not limited to skin disorder caused by microbial disease, autoimmune disease, autoinflammatory disorder, metabolic disorder, neoplastic disorder, or physical factors and/or physical insults that are mediated by inflammation. The composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing agents.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)).

a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, Pa. 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers without or with the antibody targeting specific cell type, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection using a two-compartment injector. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Materials for use in the preparation of microspheres and/or microcapsules are, e.g., biodegradable/bioerodible polymers such as polygalactin, poly-(isobutyl cyanoacrylate), poly(2-hydroxyethyl-L-glutamine), poly(lactic acid) water-soluble hydrogels. Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

Formulations for oral use include tablets containing the active ingredient(s) (e.g., cSN50, cSN50.1, cSN50.1 beta, or a NTM as set forth in SEQ ID NO: 3, SEQ ID NO: 4, SEQ ID NO: 5, SEQ ID NO: 6, SEQ ID NO: 7, SEQ ID NO: 8, and/or SEQ ID NO: 9) in a mixture with non-toxic pharmaceutically acceptable excipients. Such formulations are known to the skilled artisan. Excipients may be, for example, inert diluents or fillers (e.g., sucrose, sorbitol, sugar, mannitol, microcrystalline cellulose, starches including potato starch, calcium carbonate, sodium chloride, lactose, calcium phosphate, calcium sulfate, or sodium phosphate); granulating and disintegrating agents (e.g., cellulose derivatives including microcrystalline cellulose, starches including potato starch, croscarmellose sodium, alginates, or alginic acid); binding agents (e.g., sucrose, glucose, sorbitol, acacia, alginic acid, sodium alginate, gelatin, starch, pregelatinized starch, microcrystalline cellulose, magnesium aluminum silicate, carboxymethylcellulose sodium, methylcellulose, hydroxypropyl methylcellulose, ethylcellulose, polyvinylpyrrolidone, or polyethylene glycol); and lubricating agents, glidants, and antiadhesives (e.g., magnesium stearate, zinc stearate, stearic acid, silicas, hydrogenated vegetable oils, or talc). Other pharmaceutically acceptable excipients can be colorants, flavoring agents, plasticizers, humectants, buffering agents, and the like.

The tablets may be uncoated or they may be coated by known techniques, optionally to delay disintegration and absorption in the gastrointestinal tract and thereby providing a sustained action over a longer period. The coating may be adapted to release the active drug in a predetermined pattern (e.g., in order to achieve a controlled release formulation) or it may be adapted not to release the active drug until after passage of the stomach (enteric coating). The coating may be a sugar coating, a film coating (e.g., based on hydroxypropyl methylcellulose, methylcellulose, methyl hydroxyethyl cellulose, hydroxypropylcellulose, carboxymethylcellulose, acrylate copolymers, polyethylene glycols and/or polyvinylpyrrolidone), or an enteric coating (e.g., based on methacrylic acid copolymer, cellulose acetate phthalate, hydroxypropyl methylcellulose phthalate, hydroxypropyl methylcellulose acetate succinate, polyvinyl acetate phthalate, shellac, and/or ethylcellulose). Furthermore, a time delay material, such as, e.g., glyceryl monostearate or glyceryl distearate may be employed.

The solid tablet compositions may include a coating adapted to protect the composition from unwanted chemical changes, (e.g., chemical degradation prior to the release of the active therapeutic substance). The coating may be applied on the solid dosage form in a similar manner as that described in Swarbrick, J. and Boylan, J. C., vide supra. At least two therapeutics (e.g., a composition including cSN50, cSN50.1 or any of the NTM as set forth in SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 16, as well as any anti-microbial) may be mixed together in the tablet, or may be partitioned. In one example, the first active therapeutic is contained on the inside of the tablet, and the second active therapeutic is on the outside, such that a substantial portion of the second active therapeutic is released prior to the release of the first active therapeutic.

Formulations for oral use may also be presented as chewable tablets, or as hard gelatin capsules wherein the active ingredient is mixed with an inert solid diluent (e.g., potato starch, lactose, microcrystalline cellulose, calcium carbonate, calcium phosphate or kaolin), or as soft gelatin capsules wherein the active ingredient is mixed with water or an oil medium, for example, peanut oil, liquid paraffin, or olive oil. Powders and granulates may be prepared using the ingredients mentioned above under tablets and capsules in a conventional manner using, e.g., a mixer, a fluid bed apparatus or a spray drying equipment. Compositions as described herein can also be formulated for inhalation and topical applications. Optionally, an anti-microbial agent may be administered in combination with the NTM; such methods are known to the skilled artisan (see, e.g., Gennaro, vide supra). Combinations are expected to be advantageously synergistic.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are affected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counter indications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

6. Homology/Identity

It is understood that one way to define any known variants and derivatives or those that might arise, of the disclosed genes, proteins, herein is through defining the variants and derivatives in terms of homology to specific known sequences. For example SEQ ID NO: 2 sets forth a particular sequence of an NTM (cSN50.1). Specifically disclosed are variants of these and other genes- and proteins-derived peptide sequences herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 percent homology to the stated sequence. Those of skill in the art readily understand how to determine the homology of two proteins, peptides or nucleic acids, such as genes encoding proteins. For example, the homology can be calculated after aligning the two sequences so that the homology is at its highest level. As used herein, sequence homology is used interchangeably with sequence identity.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

7. Peptides

a) Peptide Variants

As discussed herein there are numerous variants of the NTM that are known and herein contemplated. Peptide variants and derivatives are well understood to those of skill in the art and can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Tables 4 and 5 and are referred to as conservative substitutions.

TABLE 4
Amino Acid Abbreviations

| | |
|---|---|
| Amino Acid | Abbreviations |

| | | |
|---|---|---|
| Alanine | Ala | A |
| allosoleucine | AIle | |
| Arginine | Arg | R |
| asparagine | Asn | N |
| aspartic acid | Asp | D |
| Cysteine | Cys | C |
| glutamic acid | Glu | E |
| Glutamine | Gln | Q |
| Glycine | Gly | G |
| Histidine | His | H |
| Isolelucine | Ile | I |
| Leucine | Leu | L |
| Lysine | Lys | K |
| phenylalanine | Phe | F |
| proline | Pro | P |
| pyroglutamic acid | pGlu | |
| Serine | Ser | S |
| Threonine | Thr | T |
| Tyrosine | Tyr | Y |
| Tryptophan | Trp | W |
| Valine | Val | V |

TABLE 5
Amino Acid Substitutions
Original Residue Exemplary Conservative Substitutions,
others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 5, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W. H. Freeman & Co., San Francisco pp 79-86 [1983]), acetylation of the N-terminal amine and, in some instances, amidation of the C-terminal carboxyl.

It is understood that one way to define the variants and derivatives of the disclosed protein-derived peptides herein is through defining the variants and derivatives in terms of homology/identity to specific known sequences. For example, SEQ ID NO: 2 sets forth a particular sequence of cSN50.1. Specifically disclosed are variants of these and other proteins herein disclosed which have at least, 70%, 75%, 80%, 85%, 90%, 91%, 92%, 93%, 94%, 95%, 96%, 97%, 98%, 99%, 99.1,%, 99.2%, 99.3%, 99.4%, 99.5%, 99.6%, 99.7%, 99.8%, 99.9%, or 100% sequence identity to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence.

It is understood that there are numerous amino acid and peptide analogs which can be incorporated into the disclosed compositions. For example, there are numerous D amino acids or amino acids which have a different functional substituent then the amino acids shown in Table 4 and Table 5. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site-specific way.

Molecules can be produced that resemble peptides, but which are not connected via a natural peptide linkage. For example, linkages for amino acids or amino acid analogs can include CH₂NH—, —CH₂S—, —CH₂—CH₂—, —CH═CH— (cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. I 307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appln, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. A particularly preferred non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. Stapled alpha-helical sequence of signal-sequence hydrophobic region can be used to stabilize its membrane-translocating conformation in NTM.

### C. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1: Twice Daily NTM Treatment to Shaved Backs of C57BL/6J Mice Challenged with 4 Daily Doses of Phorbol Myristoyl Acetate (PMA)

a) Reagents:

NTM (cSN50.1): Reconstituted immediately before use at 100 mg/ml in sterile water, then diluted to 1.5 mg/ml (low dose) or 5 mg/ml (high dose) with 100% EtOH. PMA (Calbiochem #524400): Stock of 16.2 mM (10 mg/ml) in DMSO diluted immediately before use to 100 μM with 100% ethanol (EtOH). 100% EtOH administered as a vehicle control for both PMA and NTM.

b) Mice:

8 week-old female C57BL/6J mice purchased from Jackson Labs acclimated for 1 week before start of experiment. 24 hours before treatment, mice were anesthetized with isoflurane and backs shaved. Randomized into 4 groups: #1=vehicle control; #2-#4 PMA+ high dose NTM; #5-#7 PMA+ low dose NTM; #8-#10 PMA+ vehicle.

c) Procedure:

20 μl vehicle (#1) or 2 nmole (1.25 μg) PMA in 20 μl (#2-#10) administered daily to each of 2 spots, both 1 cm diameter, on the back of each mouse for 4 consecutive days. Low dose NTM (30 μg/20 μl; #5-#7), high dose NTM (100 μg/20 μl; #2-#4) or vehicle (#1, #8-#10) was administered to 2 spots (20 μl/spot), each 1 cm diameter, on the back of each mouse 1 h before initial PMA challenge and every 12 h thereafter for the duration of the experiment. Mice were euthanized 1 h after 4^{th} PMA treatment and 2 skin biopsies were collected from each mouse: 1 to formalin and 1 snap-frozen in liquid nitrogen and stored at −80° C.

### 2. Example 2: Single PMA Challenge to Ears of C57BL/6J-129 Mice Treated with NTM

a) Reagents:

NTM (cSN50.1): Reconstituted immediately before use at 100 mg/ml in sterile water, then diluted to 5 mg/ml with 100% EtOH. PMA (Calbiochem #524400): Stock of 16.2 mM (10 mg/ml) in DMSO diluted immediately before use to 200 μM with 100% ethanol (EtOH). 100% EtOH administered as a vehicle control for both PMA and NTM

b) Mice:

8-9 week-old female C57BL/6J-129 mice bred in-house as wild-type controls and randomized into 3 groups: #1-#3=vehicle control; #4-#6=PMA+ vehicle; #7-#9=PMA+NTM.

c) Procedure:

20 μl vehicle (#1-#3) or 4 nmole (2.5 μg) PMA in 20 μl (#4-#9) administered to each ear, 10 μl on each side of ear. Vehicle (#1-#6) or NTM (100 μg/20 μl; #7-#9) was administered to each ear, 10 μl on each side of ear, 0.5 h before PMA challenge and at 3 h, 6 h and 8 h after challenge. Ear thickness measurements were obtained from each ear with Mitutyo calipers at baseline and 3 h, 6 h, 8 h and 24 h after PMA challenge, before treatment. Three punch biopsies of 3 mm diameter each were collected from left ear of each mouse at 8 h after PMA challenge, and 3 from right ears at 24 h after PMA challenge: 1 each to formalin, 2 snap-frozen in liquid nitrogen and stored at −80° C.

Mice were anesthetized with isoflurane for ear measurements and to collect punch biopsies at baseline, 3 h, 6 h and 8 h after PMA challenge, and sacrificed for collection of 24 h punch biopsies.

3. Analysis of the NTM Mechanism of Action in Skin Samples

Available techniques are selected from the following list: Immunohistochemistry/Immunofluorescence: Neutrophil marker and/or myeloperoxidase to quantify neutrophil infiltration, Other cell-type specific markers (e.g. macrophages, mast cells, T and B lymphocytes), Proliferation marker (e.g. PCNA, Ki67); Cytokines/chemokines (e.g. TNFα, IL-1α, IL-1β, IL-6, MCP-1) or other inflammatory mediators, and/or NF-κB and/or other inflammatory signaling proteins (e.g. phosphorylated STAT3); Quantitative RT-PCR of cytokines/chemokines or other inflammatory mediators and signaling proteins following RNA isolation from frozen skin biopsies; Immunoblot analysis of cytokines/chemokines or other inflammatory mediators and signaling proteins in skin biopsy lysates (whole cell and/or nuclear extracts); and/or direct measurement of cytokines/chemokines and other inflammatory mediators (e.g. myeloperoxidase, prostaglandin E2, leukotreine B4) in skin biopsy lysates by ELISA, cytometric bead array, radioimmune assay, or enzymatic assay.

1. A method of treating/inhibiting/reducing an inflammatory skin disease or inflammatory skin disorder caused by a microbial, allergic, autoimmune, constitutive, metabolic, neoplastic and physical insults in a subject comprising administering to the subject a therapeutically effective amount of a composition comprising a Nuclear Transport Modifier (NTM).

2. The method of claim 1, wherein the NTM comprises the sequence set forth in SEQ ID NO: 1, SEQ ID NO: 2, SEQ ID NO: 3; SEQ ID NO: 4; SEQ ID NO: 5; SEQ ID NO: 6, SEQ ID NO: 7, SEQ ID NO: 8, SEQ ID NO: 9, SEQ ID NO: 13, SEQ ID NO: 16, SEQ ID NO: 17, SEQ ID NO: 18, SEQ ID NO: 19; SEQ ID NO: 20; SEQ ID NO: 21; SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29; SEQ ID NO: 30; SEQ ID NO: 31; SEQ ID NO: 32, SEQ ID NO: 33, SEQ ID NO: 34, SEQ ID NO: 35, SEQ ID NO: 36, SEQ ID NO: 37, SEQ ID NO: 38, SEQ ID NO: 39; SEQ ID NO: 40; and/or SEQ ID NO: 41.

3. The method of claim 1, wherein the inflammatory skin disease or inflammatory skin disorder is caused by a viral infection, bacterial infection, fungal infection, or parasitic infection, autoimmune process, allergens, autoinflammatory process, metabolic process, neoplastic/oncogenic process, or physical insults that are mediated by inflammation.

4. (canceled)

5. The method of claim 3, further comprising administering to the subject an antimicrobial agent.

6. The method of claim 5, wherein the NTM composition comprises the anti-microbial agent.

7. (canceled)

8. The method of claim 3, wherein the viral infection is an infection with a virus selected from the group consisting of Herpes Simplex virus-1, Herpes Simplex virus-2, Varicella-Zoster virus, Epstein-Barr virus, Cytomegalovirus, Human Herpes virus-6, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B, Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Reovirus, Yellow fever virus, Zika virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B, Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

9. The method of claim 3, wherein the microbial insult is caused by a bacterial infection; wherein the bacteria causing the bacterial infection is not Bacillus anthracia.

10. The method of claim 9, wherein the bacterial infection is an infection with a bacteria selected from the group consisting of Mycobacterium tuberculosis, Mycobacterium bovis, Mycobacterium bovis strain BCG, BCG substrains, Mycobacterium avium, Mycobacterium intracellular, Mycobacterium africanum, Mycobacterium kansasii, Mycobacterium marinum, Mycobacterium ulcerans, Mycobacterium avium subspecies paratuberculosis, Mycobacterium leprae, Nocardia asteroides, other Nocardia species, Legionella pneumophila, other Legionella species, Acetinobacter baumanii, Salmonella typhi, Salmonella enterica, other Salmonella species, Shigella boydii, Shigella dysenteriae, Shigella sonnei, Shigella flexneri, other Shigella species, Yersinia pestis, Pasteurella haemolytica, Pasteurella multocida, other Pasteurella species, Actinobacillus pleuropneumoniae, Listeria monocytogenes, Listeria ivanovii, Brucella abortus, other Brucella species, Cowdria ruminantium, Borrelia burgdorferi, Bordetella avium, Bordetella pertussis, Bordetella bronchiseptica, Bordetella trematum, Bordetella hinzii, Bordetella pteri, Bordetella parapertussis, Bordetella ansorpii, other Bordetella species, Burkholderia mallei, Burkholderia psuedomallei, Burkholderia cepacian, Chlamydia pneumoniae, Chlamydia trachomatis, Chlamydia psittaci, Coxiella burnetii, Rickettsial species, Ehrlichia species, Staphylococcus aureus, Staphylococcus epidermidis, Streptococcus pneumoniae, Streptococcus pyogenes, Streptococcus agalactiae, Escherichia coli, Vibrio cholerae, Vibrio vulnificus, Capnocytophaga canimorsus, Campylobacter species, Neiserria meningitidis, Neiserria gonorrhea, Pseudomonas aeruginosa, other Pseudomonas species, Haemophilus influenzae, Haemophilus ducreyi, other Hemophilus species, Clostridium tetani, other Clostridium species, Yersinia enterolitica, and other Yersinia species.

11. (canceled)

12. The method of claim 3, wherein the fungal infection is an infection with a fungi selected from the group consisting of Malassezia spp, Candida albicans, Cryptococcus neoformans, Histoplasma capsulatum, Aspergillus fumigatus, Coccidiodes immitis, Paracoccidioides brasiliensis, Blastomyces dermitidis, Pneumocystis carnii, Pneumocystis jirovecii, Penicillium marneffi, and Alternaria alternata.

13. (canceled)

14. The method of claim 3, wherein the parasitic infection is an infection with a parasite selected from the group consisting of Toxoplasma gondii, Plasmodium falciparum, Plasmodium vivax, Plasmodium malariae, other Plasmodium species, Entamoeba histolytica, Naegleria fowleri, Rhinosporidium seeberi, Giardia lamblia, Enterobius vermicularis, Enterobius gregorii, Ascaris lumbricoides, Ancylostoma duodenale, Necator americanus, Cryptosporidium spp., Trypanosoma brucei, Trypanosoma cruzi, Leishmania major, other Leishmania species, Diphyllobothrium latum, Hymenolepis nana, Hymenolepis diminuta, Echinococcus granulosus, Echinococcus multilocularis, Echinococcus vogeli, Echinococcus oligarthrus, Diphyllobothrium latum, Clonorchis sinensis; Clonorchis viverrini, Fasciola hepatica, Fasciola gigantica, Dicrocoelium dendriticum, Fasciolopsis buski, Metagonimus yokogawai, Opisthorchis viverrini, Opisthorchis felineus, Clonorchis sinensis, Trichomonas vaginalis, Acanthamoeba species, Schistosoma intercalatum, Schistosoma haematobium, Schistosoma japonicum, Schistosoma mansoni, other Schistosoma species, Strongyloides stercoralis, Trichobilharzia regenti, Trichinella spiralis, Trichinella britovi, Trichinella nelsoni, Trichinella nativa, and Entamoeba histolytica.

15. The method of claim 3, wherein the inflammatory skin disease is caused by an autoimmune or autoinflammatory process.

16. The method of claim 15, wherein the autoimmune or autoinflammatory process is selected from the group consisting of Contact Dermatitis, Familial Mediterranean Fever, Graft-Versus-Host Disease, Pemphigus, Psoriasis, Rosacea, Scleroderma, Systemic Lupus Erythematosus, Achalasia, Acute disseminated encephalomyelitis, Acute motor axonal neuropathy, Addison's disease, Adiposis dolorosa, Adult Still's disease, Agammaglobulinemia, Alopecia areata, Alzheimer's disease, Amyloidosis, Ankylosing spondylitis, Anti-GBM/Anti-TBM nephritis, Antiphospholipid syndrome, Aplastic anemia, Autoimmune angioedema, Autoimmune dysautonomia, Autoimmune encephalomyelitis, Autoimmune enteropathy, Autoimmune hemolytic anemia, Autoimmune hepatitis, Autoimmune inner ear disease (AIED), Autoimmune myocarditis, Autoimmune oophoritis, Autoimmune orchitis, Autoimmune pancreatitis, Autoimmune polyendocrine syndrome, Autoimmune retinopathy, Autoimmune urticaria, Axonal & neuronal neuropathy (AMAN), Baló disease, Behcet's disease, Benign mucosal emphigoid, Bickerstaffs encephalitis, Bullous pemphigoid, Castleman disease (CD), Celiac disease, Chagas disease, Chronic fatigue syndrome, Chronic inflammatory demyelinating polyneuropathy (CIDP), Chronic recurrent multifocal osteomyelitis (CRMO), Churg-Strauss Syndrome (CSS), Eosinophilic Granulomatosis (EGPA), Cicatricial pemphigoid, Cogan's syndrome, Cold agglutinin disease, Congenital heart block, Coxsackie myocarditis, CREST syndrome, Crohn's disease, Dermatitis herpetiformis, Dermatomyositis, Devic's disease (neuromyelitis optica), Diabetes mellitus type 1, Discoid lupus, Dressler's syndrome, Endometriosis, Enthesitis, Eosinophilic esophagitis (EoE), Eosinophilic fasciitis, Erythema nodosum, Essential mixed cryoglobulinemia, Evans syndrome, Felty syndrome, Fibromyalgia, Fibrosing alveolitis, Giant cell arteritis (temporal arteritis), Giant cell myocarditis, Glomerulonephritis, Goodpasture's syndrome, Granulomatosis with Polyangiitis, Graves' disease, Guillain-Barre syndrome, Hashimoto's encephalopathy, Hashimoto's thyroiditis, Hemolytic anemia, Henoch-Schonlein purpura (HSP), Herpes gestationis or pemphigoid gestationis (PG), Hidradenitis Suppurativa (HS) (Acne Inversa), Hypogammalglobulinemia, IgA Nephropathy, IgG4-related sclerosing disease, Immune thrombocytopenic purpura (ITP), Inclusion body myositis (IBM), Interstitial cystitis (IC), Inflamatory Bowel Disease (IBD), Juvenile arthritis, Juvenile diabetes (Type 1 diabetes), Juvenile myositis (JM), Kawasaki disease, Lambert-Eaton syndrome, Leukocytoclastic vasculitis, Lichen planus, Lichen sclerosus, Ligneous conjunctivitis, Linear IgA disease (LAD), Lupus nephritis, Lupus vasculitis, Lyme disease chronic, Meniere's disease, Microscopic polyangiitis (MPA), Mixed connective tissue disease (MCTD), Mooren's ulcer, Mucha-Habermann disease, Multifocal Motor Neuropathy (MMN) or MMNCB, Multiple sclerosis, Myasthenia gravis, Myositis, Narcolepsy, Neonatal Lupus, Neuromyelitis optica, Neutropenia, Ocular cicatricial pemphigoid, Optic neuritis, Ord's thyroiditis, Palindromic rheumatism (PR), PANDAS, Paraneoplastic cerebellar degeneration (PCD), Paroxysmal nocturnal hemoglobinuria (PNH), Parry Romberg syndrome, Pars planitis (peripheral uveitis), Parsonnage-Turner syndrome, Pemphigus, Peripheral neuropathy, Perivenous encephalomyelitis, Pernicious anemia (PA), POEMS syndrome, Polyarteritis nodosa, Polyglandular syndromes type I, II, III, Polymyalgia rheumatica, Polymyositis, Postmyocardial infarction syndrome, Postpericardiotomy syndrome, Primary biliary cirrhosis, Primary sclerosing cholangitis, Progesterone dermatitis, Psoriasis, Psoriatic arthritis, Pure red cell aplasia (PRCA), Pyoderma gangrenosum, Raynaud's phenomenon, Reactive Arthritis, Reflex sympathetic dystrophy, Relapsing polychondritis, Restless legs syndrome (RLS), Retroperitoneal fibrosis, Rheumatic fever, Rheumatoid arthritis, Rheumatoid vasculitis, Sarcoidosis, Schmidt syndrome, Schnitzler syndrome, Scleritis, Scleroderma, Sjögren's syndrome, Sperm & testicular autoimmunity, Stiff person syndrome (SPS), Subacute bacterial endocarditis (SBE), Susac's syndrome, Sydenham chorea, Sympathetic ophthalmia (SO), Systemic Lupus Erythematosus, Systemic scleroderma, Takayasu's arteritis, Temporal arteritis/Giant cell arteritis, Thrombotic Thrombocytopenic purpura (TTP), Tolosa-Hunt syndrome (THS), Transverse myelitis, Type 1 diabetes, Ulcerative colitis (UC), Undifferentiated connective tissue disease (UCTD), Urticaria, Urticarial vasculitis, Uveitis, Vasculitis, Vitiligo, Vogt-Koyanagi-Harada Disease, and Wegener's granulomatosis (or Granulomatosis with Polyangiitis (GPA)).

17. (canceled)

18. The method of claim 15, wherein the autoimmune or autoinflammatory process is selected from the group consisting of Atopic Dermatitis/Eczema, Contact Dermatitis including Poison Ivy, Oak, and Sumac, Drug Hypersensitivity Reactions, Insect Bites, graft versus host disease, transplant rejection, Familial Cold Autoinflammatory Syndrome (FCAS), Muckle-Wells Syndrome (MWS), Neonatal-Onset Multisystem Inflammatory Disease (NOMID) (also known as Chronic Infantile Neurological Cutaneous Articular Syndrome (CINCA)), Familial Mediterranean Fever (FMF), Tumor Necrosis Factor (TNF)—Associated Periodic Syndrome (TRAPS), TNFRSF11A-associated hereditary fever disease (TRAPS11), Hyperimmunoglobulinemia D with Periodic Fever Syndrome (HIDS), Mevalonate Aciduria (MA), Mevalonate Kinase Deficiencies (MKD), Deficiency of Interleukin-1ß (IL-1ß) Receptor Antagonist (DIRA) (also known as Osteomyelitis, Sterile Multifocal with Periostitis Pustulosis), Majeed Syndrome, Chronic Nonbacterial Osteomyelitis (CNO), Early-Onset Inflammatory Bowel Disease, Diverticulitis, Deficiency of Interleukin-36-Receptor Antagonist (DITRA), Familial Psoriasis (PSORS2), Pustular Psoriasis (15), Pyogenic Sterile Arthritis, Pyoderma Gangrenosum, and Acne Syndrome (PAPA), Congenital sideroblastic anemia with immunodeficiency, fevers, and developmental delay (SIFD), Pediatric Granulomatous Arthritis (PGA), Familial Behçets-like Autoinflammatory Syndrome, NLRP12-Associated Periodic Fever Syndrome, Proteasome-associated Autoinflammatory Syndromes (PRAAS), Spondyloenchondrodysplasia with immune dysregulation (SPENCDI), STING-associated vasculopathy with onset in infancy (SAVI), Aicardi-Goutieres syndrome, Acute Febrile Neutrophilic Dermatosis, X-linked familial hemophagocytic lymphohistiocytosis, and Lyn kinase-associated Autoinflammatory Disease (LAID).

19. (canceled)

20. The method of claim 3, wherein the metabolic process is selected from the group consisting of Gout, Skin Aging, Xanthelasma, metabolic syndrome, diabetes mellitus, obesity, Gaucher's disease, Phenylketonuria (PKU), Maple syrup urine disease (MSUD), fatty liver, hypercholesterolemia, hypertriglyceridemia, hyperthyroidism, hypothyroidism, dyslipidemia, hypolipidemia, and galactosemia.

21. The method of claim 3, wherein metabolic skin disorder is selected from seborrheic dermatitis.

22. The method of claim 3, wherein the inflammatory skin disorder is caused by a neoplastic skin disorder.

23. The method of claim 21, wherein the neoplastic skin disorder response is selected from the group consisting of Mycosis Fungoides, Sezary Syndrome, Kaposi's Sarcoma, Adult T cell Leukemia/Lymphoma, PTEN hamartoma syndrome, Familial adenomatous polyposis, Tuberous sclerosis complex, Von Hippel-Lindau disease, ovarian teratomas, meningiomas, osteochondromas, B cell lymphoma, T cell lymphoma, Hodgkin's Disease, myeloid leukemia, bladder cancer, brain cancer, nervous system cancer, head and neck cancer, squamous cell carcinoma of head and neck, lung cancers such as small cell lung cancer and non-small cell lung cancer, neuroblastoma/glioblastoma, ovarian cancer, skin cancer, liver cancer, melanoma, squamous cell carcinomas of the mouth, throat, larynx, and lung, cervical cancer, cervical carcinoma, breast cancer, and epithelial cancer, renal cancer, genitourinary cancer, pulmonary cancer, esophageal carcinoma, head and neck carcinoma, large bowel cancer, hematopoietic cancers; testicular cancer; colon cancer, rectal cancer, prostatic cancer, and pancreatic cancer.

24. (canceled)

25. The method of claim 3, wherein the inflammatory skin disorder is caused by physical injury selected from the group consisting of, abrasion, puncture, laceration, contusion, blunt force trauma, ischemia, surgery, graft versus host disease after transplantation, bedsores, electric burn, sunburn, chemical burn, high temperature burn, low temperature burn, radiation injury, and skin aging.

26. A method of treating a wound or reducing the healing time of a wound comprising contacting the wound with a therapeutically effective amount of a composition comprising a Nuclear Transport Modifier (NTM) and antimicrobial agent.

27. (canceled)

28. A medicated adhesive bandage, wound dressing, surgical drape, suture, salve, cream, or wound adhesive comprising a therapeutically effective amount of a composition comprising a Nuclear Transport Modifier (NTM).


SEQUENCE LISTING (41 sequences)

SEQ ID NO: 1 - protein, 29 aa, Artificial Sequence
  Synthetic construct
AAVALLPAVLLALLAPCYVQRKRQKLMPC

SEQ ID NO: 2 - protein, 28 aa, Artificial Sequence
  Synthetic construct
AAVALLPAVLLALLAPCVQRKRQKLMPC

SEQ ID NO: 3 - protein, 29 aa, Artificial Sequence
  Synthetic construct
  misc_feature (1)..(4): Xaa can be any naturally occurring amino acid
  misc_feature (8)..(9): Xaa can be any naturally occurring amino acid
  misc_feature (17)..(19): Xaa can be any naturally occurring amino acid
  misc_feature (26)..(29): Xaa can be any naturally occurring amino acid
XXXXLLPXXLLALLAPXXXQRKRQKXXXX

SEQ ID NO: 4 - protein, 29 aa, Artificial Sequence
  Synthetic construct
  misc_feature (1)..(4): Xaa can be any naturally occurring amino acid
  misc_feature (8)..(9): Xaa can be any naturally occurring amino acid
  misc_feature (17)..(19): Xaa can be any naturally occurring amino acid
  misc_feature (26)..(29): Xaa can be any naturally occurring amino acid
XXXXLLPXXLLALLAPXXXQRKRQKXXXX

SEQ ID NO: 5 - protein, 28 aa, Artificial Sequence
  Synthetic construct
  misc_feature (1)..(4): Xaa can be any naturally occurring amino acid
  misc_feature (8)..(9): Xaa can be any naturally occurring amino acid
  misc_feature (18)..(19): Xaa can be any naturally occurring amino acid
  misc_feature (26)..(27): Xaa can be any naturally occurring amino acid
XXXXLLPXXLLALLAPCXXQRKRQKXXC

SEQ ID NO: 6 - protein, 29 aa, Artificial Sequence
  Synthetic construct
  misc_feature (1)..(4): Xaa can be any naturally occurring amino acid
  misc_feature (8)..(9): Xaa can be any naturally occurring amino acid
  misc_feature (17)..(19): Xaa can be any naturally occurring amino acid
  misc_feature (26)..(29): Xaa can be any naturally occurring amino acid
XXXXLLPXXLLAVLAPXXXQRKRQKXXXX

SEQ ID NO: 7 - protein, 28 aa, Artificial Sequence
  Synthetic construct
AAVALLPAVLLAVLAPCVQRKRQKLMPC

SEQ ID NO: 8 - protein, 29 aa, Artificial Sequence
  Synthetic construct
  misc_feature (1)..(4): Xaa can be any naturally occurring amino acid
  misc_feature (8)..(9): Xaa can be any naturally occurring amino acid
  misc_feature (17)..(19): Xaa can be any naturally occurring amino acid
  misc_feature (26)..(29): Xaa can be any naturally occurring amino acid
XXXXLLPXXLLAVLAPXXXQRDEQKXXXX

SEQ ID NO: 9 - protein, 28 aa, Artificial Sequence
  Synthetic construct
AAVALLPAVLLAVLAPCVQRDEQKLMPC

SEQ ID NO: 10 - protein, 10 aa, Artificial Sequence
  Synthetic construct
VQRKRQKLMP

SEQ ID NO: 11 - protein, 10 aa, Artificial Sequence
  Synthetic construct
VQRDEQKLMP

SEQ ID NO: 12 - protein, 12 aa, Artificial Sequence
  Synthetic construct
CVQRKRQKLMPC

SEQ ID NO: 13 - protein, 26 aa, Artificial Sequence
  Synthetic construct
AAVALLPAVLLALLAPVQRKRQKLMP

SEQ ID NO: 14 - protein, 7 aa, Artificial Sequence
  Synthetic construct
AAVALLP

SEQ ID NO: 15 - protein, 9 aa, Artificial Sequence
  Synthetic construct
AVLLALLAP

SEQ ID NO: 16 - protein, 28 aa, Artificial Sequence
  Synthetic construct
AAVALLPAVLLALLAPCVQRDEQKLMPC

SEQ ID NO: 17 - protein, 16 aa, Artificial Sequence
  Synthetic construct
AAVALLPAVLLALLAP

SEQ ID NO: 18 - protein, 32 aa, Artificial Sequence
  Synthetic construct
AAVALLPAVLLALLAPRRRRIEVNVELRKAKK

SEQ ID NO: 19 - protein, 34 aa, Artificial Sequence
  Synthetic construct
AAVALLPAVLLALLAPRRRRIEVNVELRKAKKDD

SEQ ID NO: 20 - protein, 34 aa, Artificial Sequence
  Synthetic construct
AAVALLPAVLLALLAPRRQRNEVVVELRKNKRDE

SEQ ID NO: 21 - protein, 34 aa, Artificial Sequence
  Synthethic constrcut
AAVALLPAVLLALLAPRRHRNEVTVELRKNKRDE

SEQ ID NO: 22 - protein, 34 aa, Artificial Sequence
  Synthetic construct
AAVALLPAVLLALLAPRRRREEEGLQLRKQKREE

SEQ ID NO: 23 - protein, 34 aa, Artificial Sequence
  Synthetic construct
AAVALLPAVLLALLAPRRRREEEGIQLRKQKREQ

SEQ ID NO: 24 - protein, 28 aa, Artificial Sequence
  Synthetic construct
AAVALLPAVLLALLAPCTEMRRRRIEVC

SEQ ID NO: 25 - protein, 37 aa, Artificial Sequence
  Synthetic construct
AAVALLPAVLLALLAPVELRKAKKDDQMLKRRNVSSF

SEQ ID NO: 26 - protein, 37 aa, Artificial Sequence
  Synthetic construct
AAVALLPAVLLALLAPVELRKNKRDEHLLKRRNVPHE

SEQ ID NO: 27 - protein, 37 aa, Artificial Sequence
  Synthetic construct
AAVALLPAVLLALLAPVELRKNKRDEHLLKKRNVPQE

SEQ ID NO: 28 - protein, 37 aa, Artificial Sequence
  Synthetic construct
AAVALLPAVLLALLAPLQLRKQKREEQLFKRRNVATA

SEQ ID NO: 29 - protein, 37 aa, Artificial Sequence
  Synthetic construct
AAVALLPAVLLALLAPIQLRKQKREQQLFKRRNVELI

SEQ ID NO: 30 - protein, 29 aa, Artificial Sequence
  Synthetic construct
AAVALLPAVLLALLAPCVELRKAKKDDQC

SEQ ID NO: 31 - protein, 29 aa, Artificial Sequence
  Synthetic construct
AAVALLPAVLLALLAPCVELRKNKRDEHC

SEQ ID NO: 32 - protein, 29 aa, Artificial Sequence
  Synthetic construct
AAVALLPAVLLALLAPCLQLRKQKREEQC

SEQ ID NO: 33 - protein, 29 aa, Artificial Sequence
  Synthetic construct
AAVALLPAVLLALLAPCIQLRKQKREQQC

SEQ ID NO: 34 - protein, 29 aa, Artificial Sequence
  Synthetic construct
AAVALLPAVLLALLAPCQMLKRRNVSSFC

SEQ ID NO: 35 - protein, 29 aa, Artificial Sequence
  Synthetic construct
AAVALLPAVLLALLAPCHLLKRRNVPHEC

SEQ ID NO: 36 - protein, 29 aa, Artificial Sequence
  Synthetic construct
AAVALLPAVLLALLAPCHLLKKRNVPQEC

SEQ ID NO: 37 - protein, 29 aa, Artificial Sequence
  Synthetic construct
AAVALLPAVLLALLAPCQLFKRRNVATAC

SEQ ID NO: 38 - protein, 29 aa, Artificial Sequence
  Synthetic construct
AAVALLPAVLLALLAPCQLFKRRNVELIC

SEQ ID NO: 39 - protein, 37 aa, Artificial Sequence
  Synthetic construct
  misc_feature (10)..(10): Xaa can be any naturally occurring amino acid
  misc_feature (13)..(14): Xaa can be any naturally occurring amino acid
AAVALLPAVXLAXXAPVELRKNKRDEHLLKRRNVPHE

SEQ ID NO: 40 - protein, 26 aa, Artificial Sequence
  Synthetic construct
AAVALLPAVLLALLAPVQRDEQKLMP

SEQ ID NO: 41 - protein, 28 aa, Artificial Sequence
  Synthetic construct
  misc_feature (10)..(10): Xaa can be any naturally occurring amino acid
  misc_feature (13)..(14): Xaa can be any naturally occurring amino acid
AAVALLPAVXLAXXAPCVQRKRQKLMPC